(19) United States
(12) Patent Application Publication
(10) Pub. No.: US 2025/0090181 A1
(43) Pub. Date: Mar. 20, 2025

(54) THERMAL THROMBECTOMY AND ATHERECTOMY SYSTEMS AND METHODS

(71) Applicant: Koya Medical, Inc., Oakland, CA (US)

(72) Inventors: Anand Doraiswamy, Dallas, TX (US); Matthew Braden Flowers, Laguna Niguel, CA (US); Andrew Schieber, Tustin, CA (US); Reghu Kunnath, Whittier, CA (US); Jarren Baldwin, Oakland, CA (US); Nathan Chu, Irvine, CA (US)

(21) Appl. No.: 18/829,022

(22) Filed: Sep. 9, 2024

Related U.S. Application Data
(60) Provisional application No. 63/583,783, filed on Sep. 19, 2023.

(51) Int. Cl. A61B 17/22

(52) U.S. Cl. CPC A61B 17/22; A61B 2017/22054; A61B 2017/22079; A61B 2017/22082; A61B 2017/22094

(57) ABSTRACT

This disclosure relates to thermal thrombectomy and atherectomy systems and methods for removing an occlusion, such as a thrombus and/or plaque from a blood vessel. The systems can include a distal balloon and a proximal balloon. The distal balloon can be positioned distal of the occlusion, and the proximal balloon can be positioned proximal of the occlusion. The system can include an irrigation catheter that directs heated fluid to the occlusion between the distal and proximal balloons. The heated fluid can soften and/or emulsify the occlusion. The system can include an aspiration catheter that can aspirate the softened and/or emulsified occlusion from the blood vessel.

### CROSS REFERENCE

This application claims the priority benefit of U.S. Provisional Application No. 63/583,783, filed Sep. 19, 2023, which is hereby incorporated by reference in its entirety. All applications for which a foreign or domestic priority is identified in the Application Data Sheet as filed with the present application are hereby incorporated by reference under 37 CFR 1.57.

### FIELD

This disclosure relates to thrombectomy and atherectomy systems and methods.

### BACKGROUND

The vascular system carries blood throughout the body. The vascular system includes arteries that distribute blood containing oxygen from the heart throughout the body and veins that carry deoxygenated blood back to the heart.

Thrombosis occurs when a thrombus (e.g., blood clot) forms within a blood vessel, whether venous or arterial. The thrombus may restrict blood flow through the blood vessel. For example, deep vein thrombosis (DVT) occurs when the thrombus forms in a deep vein, restricting blood flow back toward the heart. DVT typically develops in the lower leg, thigh, or pelvis but may also occur in other locations of the body, such as the arm. DVT can result in swelling, pain, discoloration, scaling, and/or ulcers. Additionally, a fragment (e.g., embolus) of the thrombus may break off and travel through the blood stream to the lungs, resulting in a pulmonary embolism (PE)-a potentially fatal condition.

Atherosclerosis is a chronic and progressive disease characterized by the accumulation of plaque within arterial walls. This accumulation can lead to narrowing and/or hardening of the arteries and/or restricting blood flow which can cause cardiovascular diseases, such as peripheral artery disease, coronary artery disease, and/or carotid artery disease that can lead to life-threatening complications such as heart attacks and/or strokes.

### SUMMARY

Thrombi can be removed by way of a thrombectomy procedure. A thrombectomy procedure may include navigating a guidewire with the assistance of an imaging system (e.g., fluoroscopic x-ray imaging) through the vascular system to pass through the thrombus to a distal location. A catheter can be advanced along the guidewire and through the thrombus to deploy (e.g., expand) an expandable bag distal of the thrombus. The expandable bag can be retracted proximally, scraping the inner walls of the vessel, to capture the thrombus and deposit it into the catheter for removal. The surgeon performing the thrombectomy may perform multiple passes with the expandable bag to remove the thrombus. Performing multiple passes can be time consuming and frustrating. Additionally, the likelihood of incurring damage to the vessel walls and/or valves increases with each pass. Multiple passes can increase the risk of vessel inflammation and re-thrombosis.

A thrombus can include fibrin, red blood cells, platelets, leukocytes, and neutrophil extracellular traps, and as time passes, the thrombus accumulates more collagen and fibrin content. As more collagen is accumulated, the thrombus becomes harder, and it can be difficult for guidewires, catheters, and/or expandable devices to pass through the thrombus. Additionally, it can be difficult to detach the thrombus from the vessel wall with expandable devices and/or cutting devices for removal. Accordingly, attempting to remove a thrombus, such as a hardened thrombus, can be time consuming, frustrating, and/or result in damage to the vessel wall and/or valves. Accordingly, thrombectomy solutions to ease the removal of a thrombus with less damage to vessel walls are needed.

Atherosclerosis can be addressed with an atherectomy procedure. For example, an angioplasty procedure with stenting can be performed to widen and maintain a narrowed or blocked artery. In another example, bypass surgery (e.g., coronary artery bypass grafting, peripheral artery bypass) can be performed to create a new pathway for blood flow around a blocked artery. In another example, an endarterectomy procedure (e.g., carotid endarterectomy, femoral endarterectomy) can be performed to remove plaque from the inner lining of an artery wall. However, current atherectomy procedures can be invasive, damage arterial walls, fail to remove plaque, and/or be susceptible to significant complications.

The thermal systems and methods (e.g., thermal thrombectomy and/or atherectomy systems and methods) disclosed herein may at least address the problems indicated above. The thermal systems and methods described herein may use a heated fluid to soften and/or emulsify thrombi, plaque, and/or other occlusions for aspiration, which can be advantageous over current systems and methods for thrombectomy and/or atherectomy procedures. Thrombus (e.g., acute and/or chronic), which can be made of one or more proteins and/or biopolymers, and/or plaque, which can be made of lipids, cholesterol, calcium, and/or other substances, can be softened and/or emulsified (e.g., melted, liquefied) when exposed to heat. Accordingly, when the thrombus and/or plaque is exposed to heat, the thrombus and/or plaque can soften and/or emulsify to ease penetration, detachment from vessel walls (e.g., venous or arterial), and/or aspiration, which can ease removal of the thrombus and/or plaque.

For example, the thermal systems and methods described herein can be less invasive and utilize a catheter-based approach, which may avoid open surgery and/or reduce the risk of complications. The thermal systems and methods described herein can remove more of (e.g., completely remove) a thrombus, plaque, and/or other occlusion, which can reduce repeat procedures. The thermal systems and methods can reduce damage to blood vessel walls (e.g., vein and/or arterial) in removing a thrombus, plaque and/or other occlusion, which can promote healing and reduce the risk of new thrombi and/or plaque developing. For example, the thermal systems and methods disclosed herein may obviate scraping vessel walls or reduce scraping passes to remove thrombi and/or plaque, which can decrease damage to vessel walls to improve patient outcome (e.g., reduce risk of new thrombi and/or plaque forming, complications, etc.). The thermal systems and methods disclosed herein may avoid or at least reduce the use of systemic medicaments. The thermal systems and methods disclosed herein may facilitate local removal of thrombi and/or plaque, which may preserve endothelial cells and valves. The thermal systems and methods can use a heated fluid with one or more medicaments, such as one or more bioactive agents, that can promote healing and/or reduce the risk of (e.g., prevent) new thrombi and/or plaque developing. The thermal systems and methods disclosed herein may access all anatomical blood vessel locations (e.g., below knee and smaller vessels). The thermal systems and methods disclosed herein may reduce blood loss. The thermal systems disclosed herein may be disposable (e.g., no console) or reusable.

The thermal systems and methods disclosed herein can include a first balloon and a second balloon. The first balloon can be positioned distal of an occlusion (e.g., thrombus and/or plaque) in a blood vessel. The second balloon can be positioned proximal of the occlusion. The first balloon and the second balloon can be expanded, which can isolate the segment of the blood vessel with the occlusion from the remainder of the blood vessel. The thermal systems and methods can include an irrigation device (e.g., irrigation catheter) with an irrigation aperture that can be positioned between the first balloon and the second balloon. The irrigation device can deliver a heated fluid to the occlusion to soften and/or emulsify the occlusion. The expanded first and the second balloons can impede the heated fluid and/or softened and/or emulsified occlusion from flowing beyond the first and second balloons. The thermal systems and methods can include an aspiration device (e.g., aspiration catheter) with an aspiration aperture that can be positioned between the first balloon and the second balloon. The aspiration device can aspirate the softened and/or emulsified occlusion and/or heated fluid from the blood vessel for removal. In some variants, the irrigation device and aspiration device can be combined into a single device, which can include a single device with two ports and/or openings. The flow rate, pressure, velocity, flux, irrigation opening size, and/or temperature of the heated fluid can be adjusted based on sensed conditions (e.g., sensed conditions at the occlusion site and/or in the thermal system). For example, the thermal systems and methods can include a temperature sensor that can sense a temperature at the occlusion site, which can be at the delivery of the heated fluid. Based on the sensed temperature, the thermal systems and methods can adjust the temperature of the heated fluid. The flow rate, pressure, velocity, aspiration opening size, and/or flux for aspiration can be adjusted based on sensed conditions (e.g., sensed conditions at the occlusion site and/or in the thermal system). Irrigation and aspiration can be performed simultaneously or separately (e.g., one after the other).

In some aspects, the techniques described herein relate to a thermal system for removing an occlusion from a blood vessel, the system including: an irrigation catheter including one or more apertures at a distal portion; wherein the irrigation catheter is configured to be navigated through a blood vessel to deliver a heated fluid to an occlusion to soften and/or emulsify the occlusion. The irrigation catheter can be a cannula, tube, etc.

In some aspects, the techniques described herein relate to a system, further including an aspiration catheter configured to aspirate the softened and/or emulsified occlusion.

In some aspects, the techniques described herein relate to a system, wherein the irrigation catheter and aspiration catheter are concentrically positioned.

In some aspects, the techniques described herein relate to a system, wherein the aspiration catheter is configured to include an internal pressure that aspirates in the heated fluid delivered by the irrigation catheter.

In some aspects, the techniques described herein relate to a system and 4, wherein the irrigation catheter is inside the aspiration catheter.

In some aspects, the techniques described herein relate to a system and 4, wherein the aspiration catheter is inside the irrigation catheter.

In some aspects, the techniques described herein relate to a system, wherein the one or more apertures of the irrigation catheter are disposed through a peripheral wall of the irrigation catheter.

In some aspects, the techniques described herein relate to a system, wherein the irrigation catheter includes one or more heated elements to heat the heated fluid.

In some aspects, the techniques described herein relate to a system, wherein the heated elements are disposed on the distal portion of the irrigation catheter.

In some aspects, the techniques described herein relate to a system, wherein the heated elements are disposed at the one or more apertures of the irrigation catheter.

In some aspects, the techniques described herein relate to a system, further including a fluid reservoir configured to hold the heated fluid prior to delivery.

In some aspects, the techniques described herein relate to a system, further including a reservoir heating element configured to heat the heated fluid held in the fluid reservoir.

In some aspects, the techniques described herein relate to a system, further including a crossing element configured to be heated to facilitate penetrating the occlusion.

In some aspects, the techniques described herein relate to a system, further including a distal balloon catheter including a distal balloon configured to be inflated distal of the occlusion.

In some aspects, the techniques described herein relate to a system, wherein the distal balloon catheter is configured to be advanced through the irrigation catheter to position the distal balloon distal of the irrigation catheter.

In some aspects, the techniques described herein relate to a system, further including a proximal balloon configured to be inflated proximal of the occlusion.

In some aspects, the techniques described herein relate to a system, wherein the proximal balloon is disposed on an aspiration catheter.

In some aspects, the techniques described herein relate to a system, wherein the aspiration catheter includes a working lumen through which the distal balloon catheter is configured to be advanced.

In some aspects, the techniques described herein relate to a system, wherein the irrigation catheter is configured to be advanced through the working lumen.

In some aspects, the techniques described herein relate to a system, wherein the crossing element is configured to be advanced through and distal of the distal balloon catheter.

In some aspects, the techniques described herein relate to a system, further including syringes to inflate and deflate the proximal balloon and the distal balloon.

In some aspects, the techniques described herein relate to a system, wherein the irrigation catheter further includes a distal balloon and a proximal balloon, the distal balloon configured to be inflated distal of the occlusion and the proximal balloon configured to be inflated proximal of the occlusion.

In some aspects, the techniques described herein relate to a system, wherein the irrigation catheter includes one or more aspiration apertures configured to aspirate the softened and/or emulsified occlusion.

In some aspects, the techniques described herein relate to a system, further including a temperature sensor configured to sense a temperature of the heated fluid.

In some aspects, the techniques described herein relate to a system, wherein the system is configured to adjust the temperature of the heated fluid based on the sensed temperature.

In some aspects, the techniques described herein relate to a system, wherein the occlusion includes a thrombus.

In some aspects, the techniques described herein relate to a system, wherein the occlusion includes plaque.

In some aspects, the techniques described herein relate to a system, wherein

the heated fluid is at 60-120 degrees Celsius.

In some aspects, the techniques described herein relate to a system, wherein the heated fluid includes a saline solution.

In some aspects, the techniques described herein relate to a system, wherein the heated fluid includes a thrombolytic agent.

In some aspects, the techniques described herein relate to a system, wherein the heated fluid includes urokinase.

In some aspects, the techniques described herein relate to a system, wherein the heated fluid includes an anti-inflammatory.

In some aspects, the techniques described herein relate to a system, wherein the heated fluid includes an anti-restenosis drug.

In some aspects, the techniques described herein relate to a method of softening and/or emulsifying an occlusion for removal, the method including: positioning one or more apertures of an irrigation catheter at an occlusion in a blood vessel; and delivering heated fluid through the one or more apertures of the irrigation catheter to the occlusion to soften and/or emulsify the occlusion.

In some aspects, the techniques described herein relate to a method, further including aspirating the softened and/or emulsified occlusion.

In some aspects, the techniques described herein relate to a method and 35, further including inflating a proximal balloon proximal of the occlusion.

In some aspects, the techniques described herein relate to a method, further including inflating a distal balloon distal of the occlusion.

In some aspects, the techniques described herein relate to a method, further including heating the heated fluid to 60-120 degrees Celsius.

In some aspects, the techniques described herein relate to a method, further including penetrating the occlusion with a heated element to facilitate crossover.

In some aspects, the techniques described herein relate to a method, further including sensing a temperature of the heated fluid and adjusting the temperature of the heated fluid based on the sensed temperature.

In some aspects, the techniques described herein relate to a method, further including adjusting a parameter of the heated fluid based on characteristics of the occlusion.

In some aspects, the techniques described herein relate to a method, wherein the occlusion includes a thrombus.

In some aspects, the techniques described herein relate to a method, wherein the occlusion includes plaque.

In some aspects, the techniques described herein relate to a method, wherein the heated fluid includes a saline solution.

In some aspects, the techniques described herein relate to a method, wherein the heated fluid includes a thrombolytic agent.

In some aspects, the techniques described herein relate to a method, wherein the heated fluid includes urokinase.

In some aspects, the techniques described herein relate to a method, wherein the heated fluid includes an anti-inflammatory.

In some aspects, the techniques described herein relate to a method, wherein heated the fluid includes an anti-restenosis drug.

In some aspects, the techniques described herein relate to a thermal system for removing an occlusion from a blood vessel, the system including: a multi-lumen tube including a proximal balloon and an aspiration lumen, the proximal balloon configured to be inflated proximally of an occlusion; an irrigation catheter configured to be advanced through and distally out of the multi-lumen tube to the occlusion; and a distal-balloon catheter including a distal balloon configured to be inflated distally of the occlusion, the distal-balloon catheter configured to be advanced through and distally out of the multi-lumen tube; wherein the irrigation catheter is configured to deliver heated fluid to the occlusion to soften and/or emulsify the occlusion; and wherein the aspiration lumen is configured to aspirate the softened and/or emulsified occlusion.

In some aspects, the techniques described herein relate to a thermal system, wherein the distal-balloon catheter is advanced through the irrigation catheter in the aspiration lumen.

In some aspects, the techniques described herein relate to a thermal system, wherein the irrigation catheter includes one or more apertures at a distal portion thereof.

In some aspects, the techniques described herein relate to a thermal system, wherein the one or more apertures extend through a peripheral wall of the irrigation catheter.

In some aspects, the techniques described herein relate to a thermal system, further including a heated element configured to penetrate the occlusion for crossover.

In some aspects, the techniques described herein relate to a thermal system, wherein the heated element is configured to be advanced through and distally out of the distal-balloon catheter.

In some aspects, the techniques described herein relate to a thermal system, further including syringes to inflate the proximal balloon and distal balloon.

In some aspects, the techniques described herein relate to a thermal system, further including a fluid reservoir configured to hold the heated fluid prior to delivery.

In some aspects, the techniques described herein relate to a system, further including a reservoir heating element configured to heat the heated fluid held in the fluid reservoir.

In some aspects, the techniques described herein relate to a system, further including a temperature sensor configured to sense a temperature of the heated fluid.

In some aspects, the techniques described herein relate to a system, wherein the system is configured to adjust the temperature of the heated fluid based on the sensed temperature.

In some aspects, the techniques described herein relate to a system, wherein the occlusion includes a thrombus.

In some aspects, the techniques described herein relate to a system, wherein the occlusion includes plaque.

In some aspects, the techniques described herein relate to a system, wherein the heated fluid is at 60-120 degrees Celsius.

In some aspects, the techniques described herein relate to a system, wherein the heated fluid includes a saline solution.

In some aspects, the techniques described herein relate to a system, wherein the heated fluid includes a thrombolytic agent.

In some aspects, the techniques described herein relate to a system, wherein the heated fluid includes urokinase.

In some aspects, the techniques described herein relate to a system, wherein the heated fluid includes an anti-inflammatory.

In some aspects, the techniques described herein relate to a system, wherein the heated fluid includes an anti-restenosis drug.

In some aspects, the techniques described herein relate to a system, wherein the irrigation catheter includes a heated element.

In some aspects, the techniques described herein relate to a method of softening and/or emulsifying an occlusion for removal, the method including: proximally positioning a multi-lumen tube relative to an occlusion; inflating a proximal balloon of the multi-lumen tube; advancing a distal-balloon catheter through the multi-lumen tube to position a distal balloon distal of the occlusion; inflating the distal balloon; advancing an irrigation catheter through the multi-lumen tube to the occlusion; delivering heated fluid by way of the irrigation catheter to the occlusion to soften and/or emulsify the occlusion; and aspirating the softened and/or emulsified occlusion.

In some aspects, the techniques described herein relate to a method, wherein aspirating the softened and/or emulsified occlusion is by way of an aspiration lumen of the multi-lumen tube.

In some aspects, the techniques described herein relate to a method, wherein the distal-balloon catheter is advanced through the irrigation catheter.

In some aspects, the techniques described herein relate to a method, wherein the irrigation catheter is advanced through the aspiration lumen.

In some aspects, the techniques described herein relate to a method, further including penetrating the occlusion with a heated element to facilitate crossover.

In some aspects, the techniques described herein relate to a method, further including heating the heated fluid to 60-120 degrees Celsius.

In some aspects, the techniques described herein relate to a method, further including sensing a temperature of the heated fluid and adjusting the temperature of the heated fluid based on the sensed temperature.

In some aspects, the techniques described herein relate to a method, further including adjusting a parameter of the heated fluid based on characteristics of the occlusion.

In some aspects, the techniques described herein relate to a method, wherein the occlusion includes a thrombus.

In some aspects, the techniques described herein relate to a method, wherein the occlusion includes plaque.

In some aspects, the techniques described herein relate to a method, wherein the heated fluid includes a saline solution.

In some aspects, the techniques described herein relate to a method, wherein the heated fluid includes a thrombolytic agent.

In some aspects, the techniques described herein relate to a method, wherein the heated fluid includes urokinase.

In some aspects, the techniques described herein relate to a method, wherein the heated fluid includes an anti-inflammatory.

In some aspects, the techniques described herein relate to a method, wherein heated the fluid includes an anti-restenosis drug.

In some aspects, the techniques described herein relate to a thermal system for removing an occlusion from a blood vessel, the system including: a catheter configured to deliver a heated fluid to an occlusion to soften and/or emulsify the occlusion.

In some aspects, the techniques described herein relate to a system, wherein the catheter includes a balloon that is configured to be inflated to impede flow through the blood vessel.

In some aspects, the techniques described herein relate to a system, further including a reservoir configured to hold the heated fluid.

In some aspects, the techniques described herein relate to a system, further including a reservoir heating element configured to heat the heated fluid held in the reservoir.

In some aspects, the techniques described herein relate to a system, wherein the catheter includes an opening configured to face in a proximal direction.

In some aspects, the techniques described herein relate to a system, further including an aspiration catheter configured to aspirate the softened and/or emulsified occlusion.

In some aspects, the techniques described herein relate to a system, wherein the opening of the catheter faces toward an aspiration opening of the aspiration catheter.

Neither the preceding summary nor the following detailed description purports to limit or define the scope of protection. The scope of protection is defined by the claims. Furthermore, reference is made herein to removing thrombi from veins and/or plaque from blood vessels, such as arteries. One of ordinary skill in the art will understand, after reviewing the entirety of this disclosure, that the systems and methods described herein may be applied to removing other occlusions from blood vessels of the body.

### BRIEF DESCRIPTION OF THE DRAWINGS

The abovementioned and other features of the embodiments disclosed herein are described below with reference to the drawings of the embodiments. The illustrated embodiments are intended to illustrate, but not to limit, the scope of protection. Various features of the different disclosed embodiments can be combined to form further embodiments, which are part of this disclosure.

FIG. 1A illustrates a venous system in a limb.

FIG. 1B illustrates a vein with normal blood flow.

FIG. 1C illustrates a vein with a partial occlusion.

FIG. 1D illustrates a vein with a complete occlusion.

FIG. 2A illustrates a venous system in a limb.

FIG. 2B illustrates a vein with normal blood flow.

FIG. 2C illustrates a vein with early thrombus formation.

FIG. 2D illustrates a vein with a thrombus.

FIG. 2E illustrates an embolus breaking off of the thrombus.

FIG. 3A illustrates a venous system in a limb.

FIG. 3B illustrates a thrombus in a vein of the venous system causing swelling.

FIG. 4 illustrates a table showing the changes to a thrombus over time.

FIG. 5A illustrates an acute thrombus.

FIG. 5B illustrates a chronic thrombus.

FIG. 6 illustrates a thrombus disposed in a vein.

FIG. 7A illustrates an artery without plaque.

FIG. 7B illustrates an artery with plaque.

FIG. 8A illustrates a graph showing the glass transition temperature of collagen in a thrombus and/or other thrombus forming elements and/or plaque.

FIG. 8B illustrates a graph showing the glass transition temperature of collagen in a thrombus and/or other thrombus forming elements and/or plaque.

FIG. 9 illustrates a vein with a thrombus.

FIG. 10A illustrates a catheter disposed at the thrombus.

FIG. 10B illustrates heated fluid flowing out of the catheter to the thrombus.

FIG. 10C illustrates the catheter with heated fluid flowing out as the catheter is distally advanced through the thrombus.

FIG. 10D illustrates the catheter with heated fluid flowing out as the catheter is distally advanced through the thrombus.

FIG. 10E illustrates the catheter crossed over the thrombus with a distal portion of the catheter distal of the thrombus.

FIG. 11A illustrates an irrigation catheter for heated fluid disposed inside of an aspiration catheter.

FIG. 11B illustrates an irrigation catheter with heated elements to heat fluid and the irrigation catheter disposed inside of an aspiration catheter.

FIG. 11C illustrates an irrigation catheter disposed inside of an aspiration catheter with a temperature sensor.

FIG. 11D illustrates an irrigation catheter for heated fluid with a closed distal end and one or more lateral openings disposed inside of an aspiration catheter.

FIG. 12A illustrates a catheter disposed at a thrombus.

FIG. 12B illustrates a heated element deployed distally from the catheter to facilitate crossing the thrombus using heat.

FIG. 12C illustrates the heated element and catheter crossed over the thrombus.

FIG. 12D illustrates the heated element retracted proximally from the catheter with the catheter remaining distal of the thrombus.

FIG. 13A illustrates the catheter being retracted proximally to expose a first balloon of an irrigation and aspiration catheter distal of the thrombus.

FIG. 13B illustrates the catheter being retracted proximally to expose one or more apertures of the irrigation and aspiration catheter.

FIG. 13C illustrates the catheter being retracted proximally to expose a second balloon of the irrigation and aspiration catheter proximal of the thrombus.

FIG. 14A illustrates the first and second balloons of the irrigation and aspiration catheter being inflated.

FIG. 14B illustrates the first and second balloons of the irrigation and aspiration catheter inflated.

FIG. 15A illustrates heated fluid flowing out of the one or more apertures disposed between the first and second balloons to soften and/or emulsify the thrombus.

FIG. 15B illustrates heated fluid flowing out of the one or more apertures disposed between the first and second balloons of the irrigation and aspiration catheter to soften and/or emulsify the thrombus.

FIG. 15C illustrates one or more apertures of the irrigation and aspiration catheter irrigating heated fluid.

FIG. 15D illustrates an aperture of the irrigation and aspiration catheter irrigating heated fluid.

FIG. 16A illustrates the softened and/or emulsified thrombus being aspirated through the one or more apertures of the irrigation and aspiration catheter.

FIG. 16B illustrates the softened and/or emulsified thrombus being aspirated through the one or more apertures of the irrigation and aspiration catheter.

FIG. 16C illustrates the softened and/or emulsified thrombus being aspirated through the one or more apertures of the irrigation and aspiration catheter.

FIG. 16D illustrates the thrombus aspirated from the vein.

FIG. 16E illustrates the one or more apertures of the irrigation and aspiration catheter aspirating fluid and/or softened and/or emulsified thrombus.

FIG. 16F illustrates one aperture of the irrigation and aspiration catheter aspirating fluid and/or softened and/or emulsified thrombus.

FIG. 17A illustrates a cross-sectional view of an example catheter configuration to deliver and aspirate heated fluid from an occlusion site.

FIG. 17B illustrates a cross-sectional view of an example catheter configuration to deliver and aspirate heated fluid from an occlusion site.

FIG. 18A illustrates an example catheter of a thermal system to deliver heated fluid to an occlusion site and aspirate the softened and/or emulsified occlusion.

FIG. 18B illustrates an example catheter of a thermal system to deliver heated fluid to an occlusion site and aspirate the softened and/or emulsified occlusion.

FIG. 19A illustrates a balloon catheter with a first balloon distal of the thrombus and a second balloon proximal of the thrombus being inflated.

FIG. 19B illustrates the balloon catheter with the first balloon distal of the thrombus and the second balloon proximal of the thrombus inflated to isolate the segment of the vein with the thrombus.

FIGS. 20A, 20B, 20C, 20D, 20E, 20F, and 20G illustrate heated fluid being delivered with an irrigation catheter to soften and/or emulsify the thrombus and an aspiration catheter aspirating the heated fluid and/or softened and/or emulsified thrombus as the irrigation catheter and aspiration catheter are distally advanced.

FIG. 20H illustrates the irrigation catheter and aspiration catheter being retracted proximally into a catheter.

FIG. 20I illustrates the first and second balloons of the balloon catheter being deflated.

FIG. 21A illustrates a cross-sectional view of an example catheter configuration to deliver and aspirate heated fluid from an occlusion site

FIG. 21B illustrates a cross-sectional view of an example catheter configuration to deliver and aspirate heated fluid from an occlusion site.

FIG. 22A illustrates an example catheter of a thermal system to deliver heated fluid to an occlusion site and aspirate the softened and/or emulsified occlusion

FIG. 22B illustrates the example catheter of FIG. 22A with a heated element deployed distally out of the catheter.

FIG. 23A illustrates catheters of a thermal system positioned to deliver heated fluid to a thrombus and aspirate the softened and/or emulsified thrombus.

FIG. 23B illustrates a cross-sectional view of a two-lumen catheter.

FIG. 23C illustrates a cross-sectional view of a three-lumen catheter.

FIG. 24A illustrates a catheter of a thermal system positioned to deliver heated fluid to a thrombus and aspirate the softened and/or emulsified thrombus.

FIG. 24B illustrates the catheter of the thermal system of FIG. 24A with a plurality of apertures to deliver heated fluid to the thrombus and/or aspirate the softened and/or emulsified thrombus.

FIG. 25 illustrates a cross-sectional view of the catheter of FIGS. 24A and 24B.

FIG. 26A illustrates a schematic of a thermal system.

FIG. 26B illustrates a schematic of a pneumatic system of the thermal system.

FIG. 26C illustrates a schematic of a balloon control system of the thermal system.

FIG. 27A illustrates a catheter of a thermal system.

FIG. 27B illustrates components of the catheter of FIG. 27A.

FIG. 27C illustrates a distal end of the catheter of the thermal system of FIG. 27A.

FIG. 27D illustrates a distal end of the catheter of the thermal system of FIG. 27A.

FIG. 27E illustrates a cross-sectional view of catheter of the thermal system of FIG. 27A.

FIG. 28 illustrates a multi-lumen tube coupled to a Y connector.

FIG. 29A illustrates the multi-lumen tube of FIG. 28.

FIG. 29B illustrates a cross-section of the multi-lumen tube of FIG. 28.

FIG. 29C illustrates the multi-lumen tube of FIG. 28 coupled to a Y connector coupled to a syringe.

FIG. 29D illustrates a cross-section of the catheter of the distal balloon.

FIG. 29E illustrates a cross-section of the irrigation catheter.

FIG. 29F illustrates a cross-section of the multi-lumen tube.

FIG. 29G illustrates a table of example dimensions of features of the thermal system. FIG. 29H schematically illustrates an example cross section that corresponds to the table of example dimensions provided in FIG. 29G.

FIG. 30A illustrates an irrigation catheter coupled to a Y connector.

FIG. 30B illustrates the irrigation catheter of FIG. 30A without the Y connector.

FIG. 30C illustrates a cross-section view of the irrigation catheter.

FIG. 31A illustrates a balloon catheter coupled to a Y connector.

FIG. 31B illustrates the balloon catheter of FIG. 31A without the Y connector.

FIG. 32A illustrates the balloon catheter of FIG. 31A without the Y connector.

FIG. 32B illustrates a cross-section of the catheter of the distal balloon.

FIG. 32C illustrates a heated element advanced distally out of the lumen of a distal balloon catheter.

FIG. 33A illustrates a cross-section of the connection between the Y connector with the aspiration port and the Y connector with the port for filling the proximal balloon.

FIG. 33B illustrates a cross-section of the multi-lumen tube.

FIG. 33C illustrates a cross-section of the connection between the Y connector with the irrigation port and the Y connector with the aspiration port.

FIG. 34A illustrates distal ends of irrigation and aspiration catheters.

FIG. 34B illustrates distal ends of irrigation and aspiration catheters.

FIG. 34C illustrates another view of the irrigation catheter of FIG. 34B.

FIG. 35A illustrates a fluid jet system.

FIG. 35B illustrate an irrigation jet catheter delivering a jet of fluid to fragment a thrombus.

FIG. 35C illustrates an aspiration catheter aspirating fragments of a thrombus.

FIG. 35D illustrates an irrigation jet catheters positioned to fragment a thrombus.

FIG. 35E illustrates the irrigation jet catheters with temperature sensors.

### DETAILED DESCRIPTION

Although certain embodiments and examples are described below, this disclosure extends beyond the specifically disclosed embodiments and/or uses and obvious modifications and equivalents thereof. Thus, it is intended that the scope of this disclosure should not be limited by any particular embodiments described below. Furthermore, this disclosure describes many embodiments in reference to veins and arteries; the systems and methods described in relation to veins can be applied to arteries and those described in relation to arteries can be applied to veins.

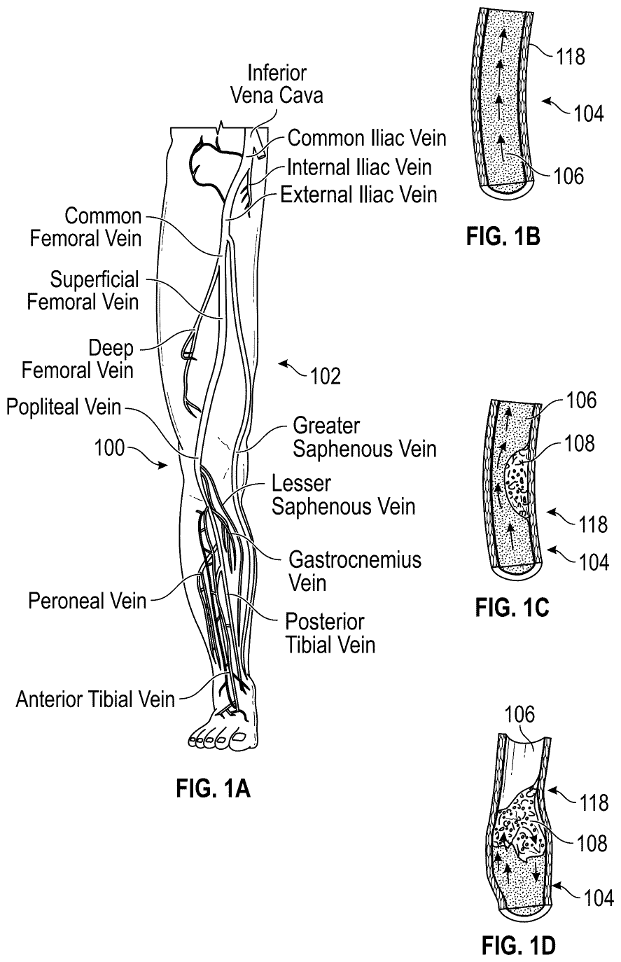

FIG. 1A illustrates a limb 100 (e.g., leg, arm) of a person. The limb 100 includes a venous system 102 that carries deoxygenated blood from the limb 100 back to the heart.

FIG. 1B illustrates a cross-section of a portion of a vein 104 of the venous system 102 with normal blood flow. The vein 104 includes a flow path 106 for the deoxygenated blood bounded by the wall 118 of the vein 104. A thrombus 108 may develop within a vein 104, as illustrated in FIG. 1C, to partially restrict (e.g., impede, occlude, etc.) blood flow through the 106. The thrombus 108 may form on the wall 118 of the vein 104, which may include a valve. The wall 118 may from along the wall 118 for a variety of reasons, which may include damage to the wall 118, inactivity, diet, and/or other reasons. The thrombus 108 may grow in size to completely block (e.g., impede, occlude, etc.) blood flow through the flow path 106, as shown in FIG. 1D. The occlusion of the blood flow can result in swelling, pain, and/or discoloration of the limb 100. The removal of thrombi can relieve a patient of the foregoing symptoms as well as prevent or at least slow further symptoms from developing.

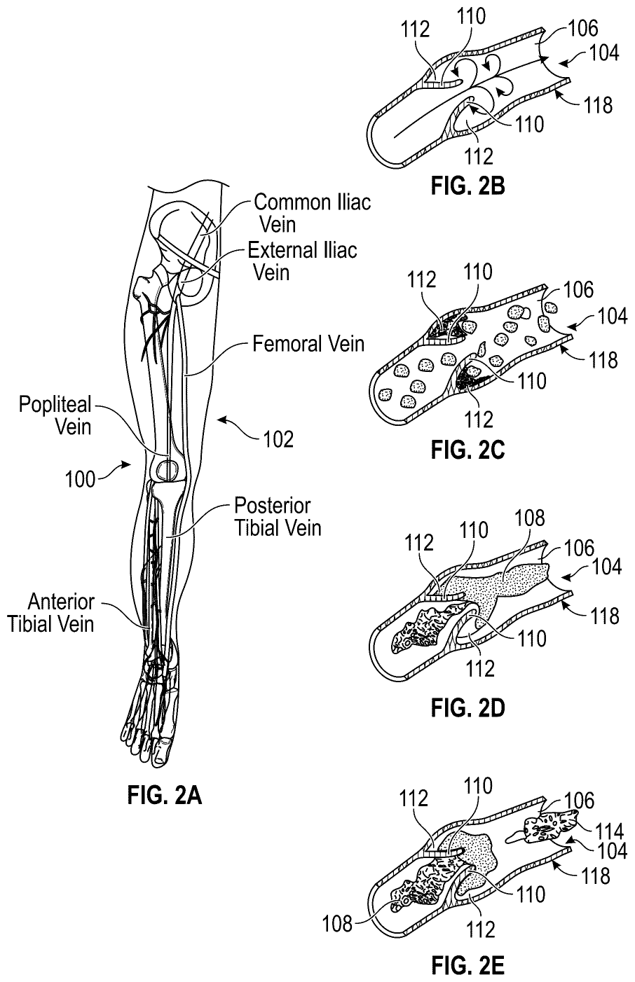

FIG. 2A illustrates a limb 100 (e.g., leg, arm) of a person. The limb 100 includes venous system 102 that carries deoxygenated blood from the limb 100 back to the heart.

FIG. 2B illustrates a cross-section of a portion of a vein 104 of the venous system 102 with normal blood flow. The vein 104 includes a flow path 106 for the deoxygenated blood bounded by the wall 118 of the vein 104. The vein 104 may include valves 110. The valves 110 may open to allow blood to flow toward the heart and close to prevent backflow. Pockets 112 can be disposed downstream of the valves 110 between the valves 110 and the wall 118 of the vein 104. As shown, a portion of blood in the flow path 106 may flow into the pockets 112. A thrombus may begin to develop on the wall 118 of the vein 104, valves 110, and/or in the pockets 112, as shown in FIG. 2C, until a thrombus 108 is formed, as illustrated in FIG. 2D. The thrombus 108 may fill the flow path 106, which can include extending around the valves 110 and into the pockets 112. The thrombus 108 may prevent proper function of the valves 110. The thrombus 108 may partially or completely block blood flow through the vein 104. In some instances, an embolus 114 may break off from the thrombus 108, as illustrated in FIG. 2E. The embolus 114 may travel to other regions of the body, which can include traveling to the lungs and result in a pulmonary embolism (PE)-a potentially lethal condition. Accordingly, the removal of thrombi may help a patient avoid a lethal condition.

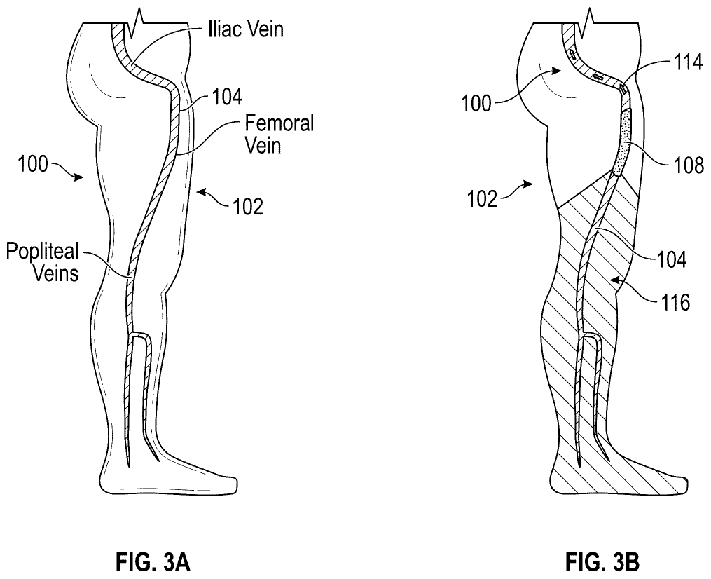

FIG. 3A illustrates a limb 100 (e.g., leg, arm) of a person. The limb 100 includes a venous system 102 with multiple veins 104. As shown in FIG. 3B, a thrombus 108 may form in the vein 104 to block blood flow. The blocked blood flow may prevent drainage of blood from the limb 100 and cause swelling of the limb 100 below the thrombus 108 as indicated by portion 116. As detailed herein, an embolus 114 may separate from the thrombus 108 and travel to other regions of the body, which can include traveling to the lungs and result in a pulmonary embolism (PE).

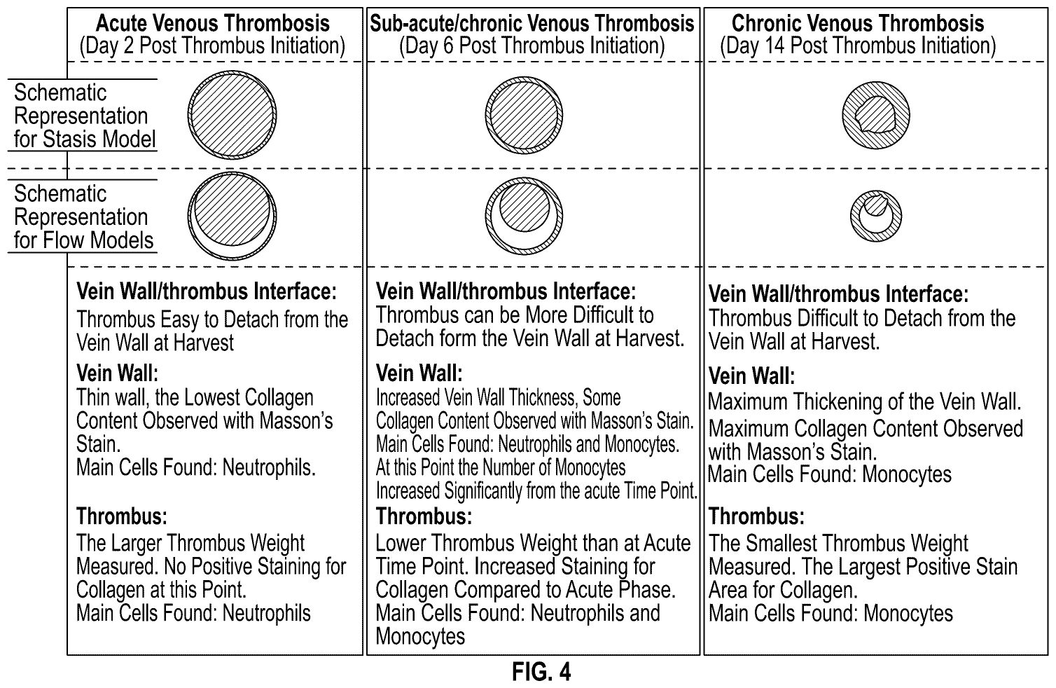

As a thrombus ages, the characteristics of the proximate portion of the vein and the thrombus itself may change, as indicated in the table shown in FIG. 4.

For example, initially after formation (e.g., two days post thrombus initiation), a thrombus may be referred to as an acute thrombus. The vein wall proximate the acute thrombus may be thin and have a low collagen content. The main cells found in the vein wall proximate the acute thrombus may be neutrophils. The acute thrombus itself may have no or relatively little collagen. The main cells found in the acute thrombus may be neutrophils. The acute thrombus may be readily detached from the vein wall and/or penetrated.

After some additional time (e.g., six days post thrombus initiation), a thrombus may be referred to as a sub-acute/chronic thrombus. The vein wall proximate the sub-acute/chronic thrombus may be thickened and have a higher collagen content compared to the acute thrombus period. The main cells found in the vein wall proximate the sub-acute/chronic thrombus may be neutrophils and monocytes with the quantity of monocytes significantly increased compared to the acute thrombus period. The sub-acute/chronic thrombus itself may have an increased collagen content compared to the acute thrombus. The main cells found in the sub-acute/chronic thrombus may be neutrophils and monocytes. The sub-acute/chronic thrombus may be more difficult to detach from the vein wall compared to the acute thrombus and/or more difficult to penetrate. The sub-acute/chronic thrombus may have a lower weight compared to the acute thrombus.

After some additional time (e.g., fourteen days post thrombus initiation), a thrombus may be referred to as a chronic thrombus. The vein wall proximate the chronic thrombus may be thickened and have a higher collagen content compared to the sub-acute/chronic thrombus period. The main cells found in the vein wall proximate the chronic thrombus may be monocytes. The chronic thrombus may have an increased collagen content compared to the sub-acute/chronic thrombus. The main cells found in the chronic thrombus may be monocytes. The chronic thrombus may be more difficult to detach from the vein wall compared to the sub-acute/chronic thrombus and/or more difficult to penetrate. The chronic thrombus may have a lower weight compared to the sub-acute/chronic thrombus.

The hardness of a thrombus may increase over time, which may be due to the increase in collagen content. For example, a chronic thrombus may be harder than a sub-acute/chronic thrombus which may be harder than an acute thrombus. A harder thrombus may be more difficult to penetrate, which can make removal more difficult. Additionally, as indicated above, a chronic thrombus may be harder to detach from the vein wall compared to the sub-acute/chronic thrombus which may be harder to detach from the vein wall compared to the acute thrombus.

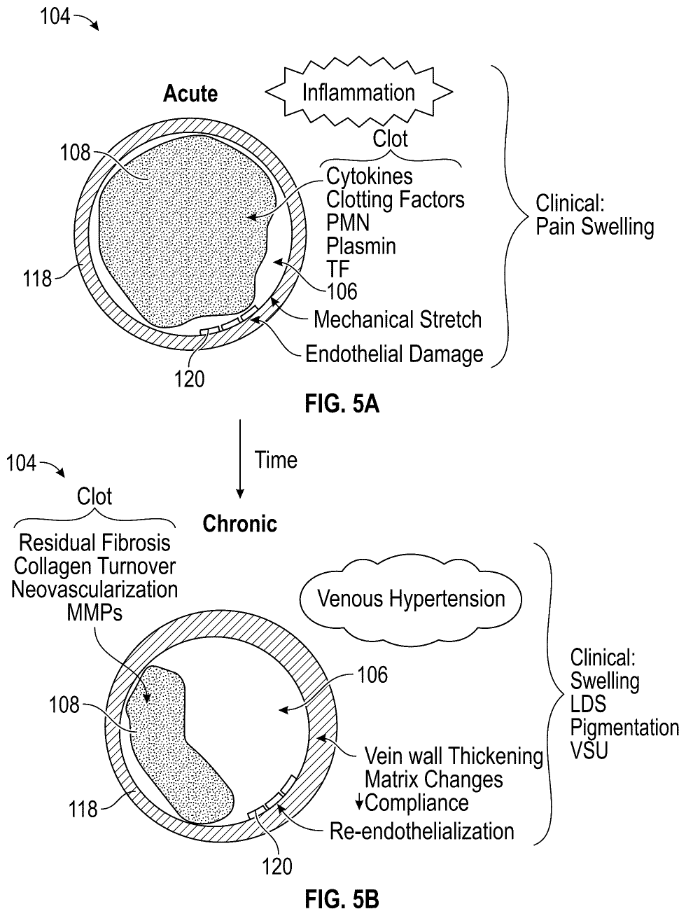

As shown in FIG. 5A, an acute thrombus may be accompanied by inflammation of the proximate vein wall as well as stretching of the proximate vein wall. The acute thrombus may also damage the endothelial cells along the vein wall. The patient may experience pain and swelling in the region of the body with the acute thrombus. The acute thrombus may progress into a chronic thrombus over time. The chronic thrombus may be accompanied by venous hypertension, thickening of the vein wall, and matrix changes within the vein wall. The patient may at least experience swelling and pigmentation in the region of the body with the chronic thrombus.

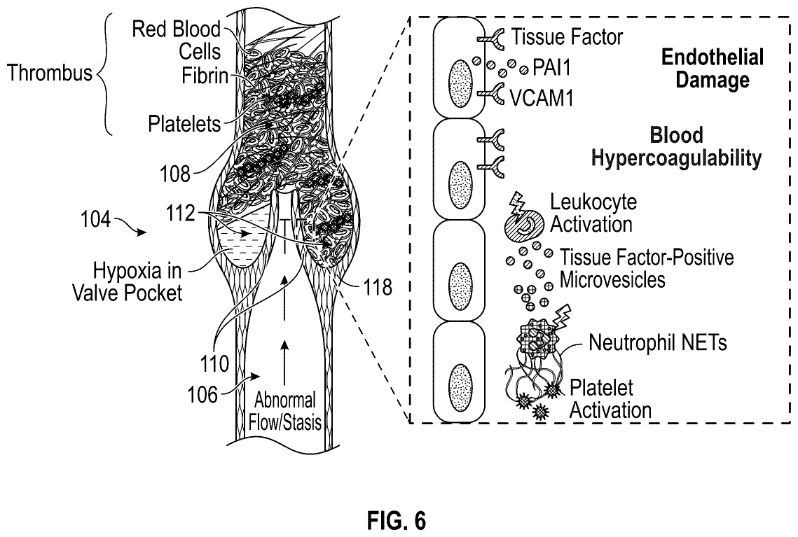

FIG. 6 illustrates another example section of a vein 104 with a thrombus 108. The thrombus 108 is disposed against the wall 118 and valve 110 of the vein 104. The thrombus 108 is disposed in the pockets 112. As illustrated, the thrombus 108 may result in hypoxia in the pockets 112 and surrounding tissue. During a thrombectomy procedure, the thrombus 108 is penetrated, but as explained, the thrombus 108 may become harder overtime making penetration more difficult. Additionally, the thrombus 108 may be increasingly harder to detach from the wall 118 and/or valves 110 as time passes. Accordingly, at least due to the foregoing reasons, a surgeon may need to perform multiple passes with a thrombectomy device to remove the thrombus. With each pass, the wall 118 may be scrapped in an effort to remove the thrombus. The likelihood of damaging the wall 118 and/or valves 110 of the vein 104 increases with the number of passes. The thermal thrombectomy systems and methods disclosed herein may reduce the number of passes, which may include reducing to a single pass, to remove a thrombus, decreasing the likelihood of damage to the wall 118 and/or valves 110 which may decrease the risk of re-thrombosis. The thermal thrombectomy systems and methods disclosed herein may reduce the overall ease and/or time of a procedure, which may lower the risk of complications and improve efficiency.

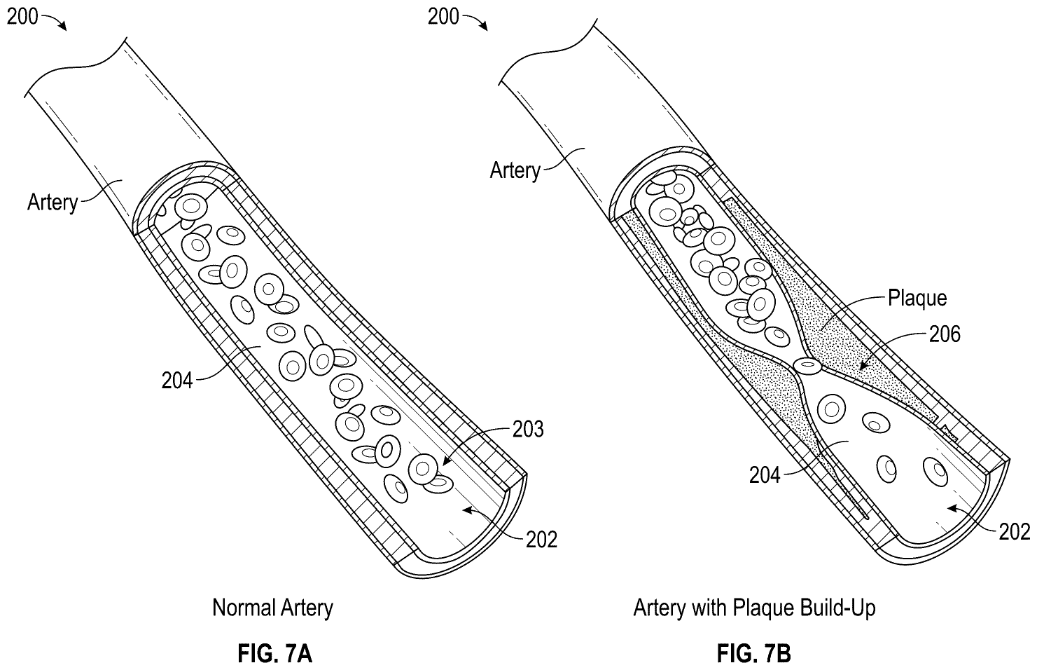

FIG. 7A illustrates an example section of an artery 200. As shown, the artery 200 includes a flow path 202 through which red blood cells 203 can travel. The artery 200 can include a wall 204, which can be smooth, surrounding the flow path 202. FIG. 7B illustrates the example section of the artery 200 with plaque 206. Plaque 206 can accumulate on the wall 204 of the artery 200. As the plaque 206 accumulates, a partial and/or complete occlusion can develop, which can impede and/or even fully stop flow through the artery 200 (e.g., impede and/or even fully stop the flow of red blood cells 203), which can cause negative cardiovascular events that can be life threatening.

As described herein, the thermal systems and methods disclosed herein may include heating an occlusion, such as a thrombus and/or plaque. Heating a thrombus may ease penetration of the thrombus. Heating the thrombus and/or plaque may ease breaking the thrombus and/or plaque apart. Heating the thrombus and/or plaque may ease detachment of the thrombus and/or plaque from the wall of the blood vessel. Heating the thrombus may soften and/or emulsify (e.g., melt, liquify) one or more proteins (e.g., collagen) and/or other thrombus forming elements (e.g., biopolymers) detailed herein. Heating plaque may soften and/or emulsify (e.g., melt, liquify) the plaque, which can be made of lipids, cholesterol, calcium, and/or other substances. The thrombus and/or plaque can soften and/or emulsify when heated to a temperature of 60-120 degrees Celsius (e.g., 60, 70, 80, 90, 100, 110, 120 degrees Celsius), 60-80 degrees Celsius, 65-75 degrees Celsius, and/or 60-70 degrees Celsius. Accordingly, the heated fluid described herein may be heated to 60-120 degrees Celsius (e.g., 60, 70, 80, 90, 100, 110, 120 degrees Celsius), 60-80 degrees Celsius, 65-75 degrees Celsius, 60-70 degrees Celsius, and/or other temperatures. The heated fluid can be heated to 64-70 degrees Celsius, 66-70 degrees Celsius, and/or 68-70 degrees Celsius. The heated fluid can be heated to 70-80 degrees Celsius, 70-72 degree Celsius, 72-74 degree Celsius, 74-76 degree Celsius, 76-78 degree Celsius, and/or 78-80 degrees Celsius. The heated fluid can be heated to 64-66 degrees Celsius and/or 66-68 degrees Celsius. The heated fluid can be heated to about 80 degrees Celsius. The heated fluid can be heated to at least any temperature within the foregoing ranges. The temperature of the heated fluid can be modulated and/or controlled based on temperatures at the occlusion site. For example, the thermal systems and methods disclosed herein can use one or more temperature sensors (e.g., thermocouples) to sense temperatures at the occlusion site, and based on the sensed temperatures, modulate and/or control the temperature of the heated fluid. The systems and/or methods described herein can include insulation to protect anatomy of the patient from heat.

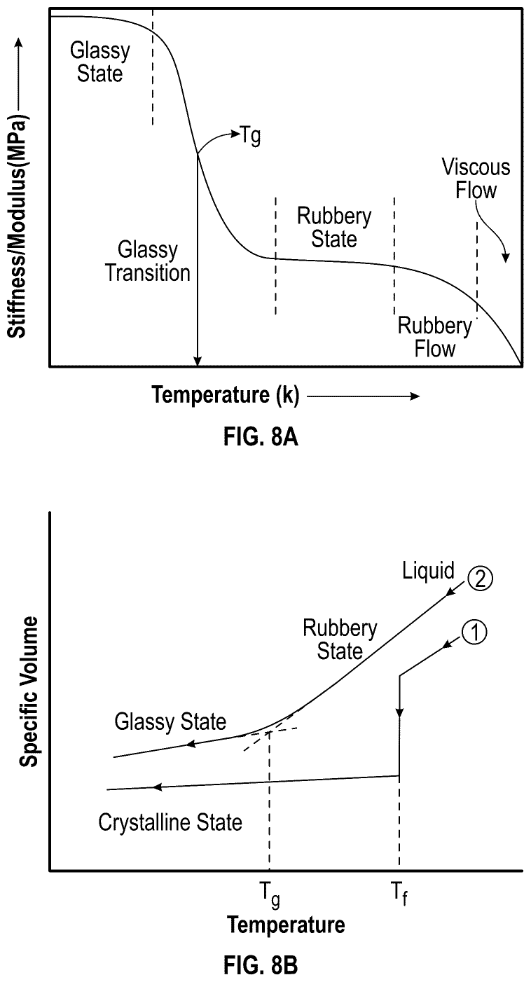

Heating the thrombus and/or plaque may facilitate a glass transition of the thrombus and/or plaque (e.g., soften). For example, FIG. 8A illustrates an example graph representing the stiffness/modulus (MPa) against temperature (k) of thrombi and/or plaque. As shown, at a glass transition temperature (Tg), the one or more proteins (e.g., collagen) and/or other thrombus forming elements may reach a glass transition where they may start flowing more readily in the glassy state (solid to liquid). As shown, at a glass transition temperature (Tg), the lipids, cholesterol, calcium, and/or other plaque forming elements may reach a glass transition where they may start flowing more readily in the glassy state (solid to liquid). FIG. 8B illustrates an example graph representing the specific volume of thrombus and/or plaque forming elements against temperature. Once again, at a glass transition temperature (Tg), the thrombus and/or plaque may reach a glass transition. When heated to the glass transition temperature, the thrombus forming elements and/or plaque forming elements may change from a first state (e.g., hard state) to a second state softer than the first state. When heated to the glass transition temperature, the thrombus forming elements and/or plaque forming elements may decrease in viscosity.

The thermal systems and methods described herein may heat thrombi and/or plaque to a temperature sufficient to facilitate a glass transition, which can include softening and/or emulsifying (e.g., melting, liquifying). The thermal systems and methods described herein may heat thrombi and/or plaque to decrease viscosity. The temperature to soften the thrombus and/or plaque may be the glass transition temperature of the thrombus and/or plaque. The temperature to emulsify (e.g., melt, liquify) the thrombus and/or plaque may be higher than the transition temperature. The temperatures to soften the thrombi and the plaque may be different. The temperatures to emulsify the thrombus and plaque may be different. Thrombi and/or plaque can be heterogeneous. Different regions of a thrombus and/or plaque can emulsify at different rates at different temperatures. In some variants, altering a temperature of the applied heat based on the characteristics of the thrombus and/or plaque can be beneficial. For example, it may be beneficial to raise a temperature of the applied heat when interfacing with a region of a thrombus with chronic characteristics compared to when interfacing with a region of the thrombus with acute characteristics. In some variants, the systems and methods described herein can alter the temperature of the applied heat based on the characteristics of the thrombus and/or plaque with which the heated fluid is interfacing.

As described herein, the thermal system and methods described herein may deliver heated fluid to a thrombus and/or plaque to soften and/or emulsify the thrombus and/or plaque. The heated fluid can be heated by way of a variety of techniques which can at least include indirect or direct heat with one or more energy sources, which may at least include heat, radio frequency, laser, electricity (e.g., current), resistive heating, inductive heating, ultrasound, heated fluid, nuclear, and/or others. The fluid can held in a reservoir (e.g., tank, bag, bottle, container, syringe, chamber, vessel, compartment, etc.) prior to delivery. The fluid can be heated in the reservoir, during transport to the occlusion site (e.g., plaque, thrombus, and/or other occlusion), and/or at delivery to the occlusion site. The fluid can include a variety of constituents. The fluid can be a saline solution such as a balanced salt solution. The fluid can include one or more medicaments, which can include a thrombolytic agent, such as tissue plasminogen activator (tPA) and/or urokinase that can assist in dissolving the thrombus and/or plaque, and/or a bioactive agent, such as an anti-inflammatory and/or an anti-restenosis drug that can promote healing and/or prevent the formation of new thrombi and/or plaque. The heated fluid can be used to penetrate and/or cross over a thrombus, which can include by way of a fluid jet. The heated fluid can be used to soften and/or emulsify thrombi and/or plaque for aspiration from a blood vessel. The temperature, pressure, and/or flow rate of the heated fluid can be adjusted based on sensed conditions at the occlusion site.

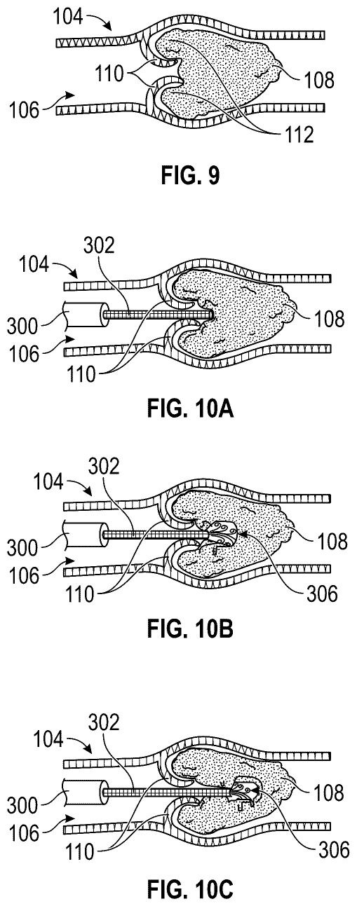

FIG. 9 illustrates a thrombus 108 in a vein 104. As shown, the thrombus 108 is disposed downstream of the valve 110 in the pocket 112, which can impede function of the valve 110 and/or impede blood flow through the vein 104.

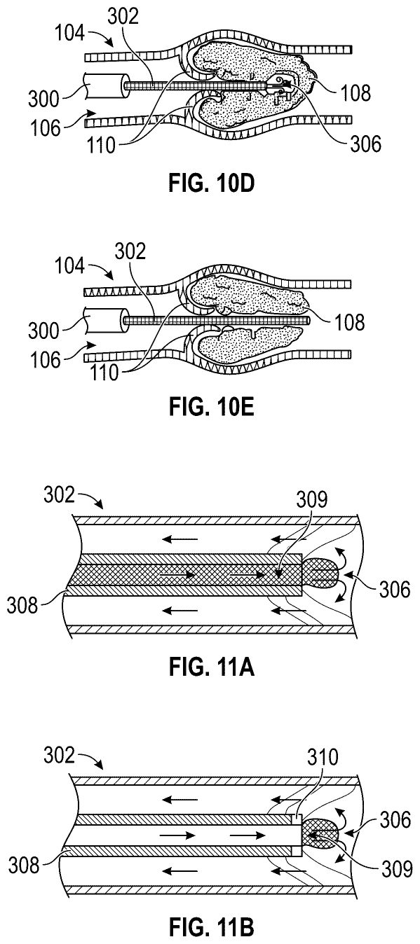

FIGS. 10A-10E illustrate a method of crossing a thrombus with heated fluid. As illustrated in FIG. 10A, a catheter 300 (e.g., working catheter, aspiration catheter) can be navigated to an occlusion site (e.g., proximal of an occlusion site). In some variants, a guidewire can be advanced through the blood vessel system (e.g., venous or arterial) to the occlusion site, which can be performed using fluoroscopic guidance. The catheter 300 can be advanced over the guidewire to the occlusion site. The guidewire can be removed, leaving the catheter 300 in place. A catheter 302 (e.g., inner catheter, aspiration catheter) can be advanced distal of the catheter 300 to the thrombus 108.

As illustrated in FIG. 10B, a fluid jet 306, which can be a stream of fluid, flow of fluid, etc., can be directed out of the distal end of the catheter 302 to interface with the thrombus 108. The fluid jet 306 can be heated to any of the temperatures described herein. The heat from the fluid jet 306 can ease penetration and/or crossing over of the thrombus 108. The heat from the fluid jet 306 can soften and/or emulsify a core (e.g., central portion) of the thrombus 108. The fluid can be heated in a reservoir (e.g., bag, tank, bottle, chamber, container, etc.). In some variants, the reservoir can be an IV bag. In some variants, the reservoir can be positioned proximate a heating element such as a pad to heat the fluid in the reservoir. In some variants, the fluid can be heated as the fluid travels distally through the catheter 302, which can be by way of one or more heating elements. In some variants, the fluid can be heated at the distal end of the catheter 302, which can be by way of one or more heating elements disposed at the distal end of the catheter 302. As illustrated in FIGS. 10C and 10D, the catheter 302 can be advanced distally as the fluid jet 306 penetrates through the thrombus 108 until crossing over, as illustrated in FIG. 10E. In some variants, the catheter 302 can include one or more heating elements to directly heat the thrombus 108. In some variants, the fluid jet 306 can cease when the catheter 302 crosses over the thrombus 108. In some variants, the catheter 300 can include a lumen to aspirate the heated fluid and/or softened and/or emulsified thrombus 108. In some variants, the catheter 302 can include a lumen to aspirate the heated fluid and/or softened and/or emulsified thrombus 108. In some variants, aspiration can be facilitate by way of a pump (peristaltic pump), vacuum (e.g., vacuum cannister), etc. The aspiration of the heated fluid can form a plume (e.g., controlled area) for treatment with the heated fluid.

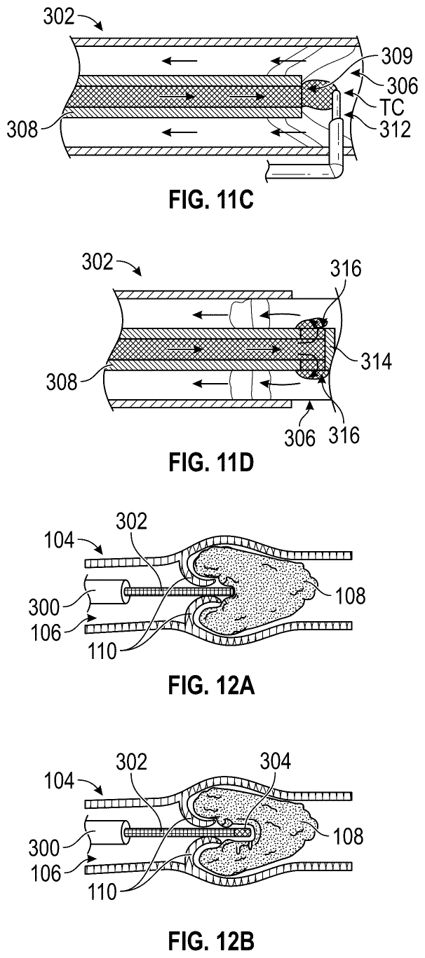

For example, as illustrated in FIG. 11A, an irrigation catheter 308 can be disposed inside the catheter 302. The irrigation catheter 308 can include an open distal end 309 through which the heated fluid jet 306 can flow. The catheter 302 can aspirate the heated fluid and/or softened and/or emulsified thrombus 108. In some variants, the open distal end 309 of the irrigation catheter 308 may be maintained proximate of the distal end of the catheter 302, which can help to localize the fluid jet 306. In some variants, the open distal end 309 of the irrigation catheter 308 can be advanced distal of the catheter 302. In some variants, a pressure in the catheter 302 can be reduced to facilitate the flow of fluid jet 306 back into the catheter 302 (e.g., flow of fluid in fluid jet 306 can reverse back and into catheter 302), which can control the plume (e.g., softening and/or emulsification zone). In some variants, the distal end of the catheter 302 and/or irrigation catheter 308 can be at an angle (e.g., less than 15, 15, 30, 45, 60, 75, 90, or more than 90 degrees) relative to the longitudinal axis of the catheter 302 and/or irrigation catheter 308, which can avoid the fluid jet 306 jetting forward. In some variants, the irrigation catheter 308 can include a curve to face a distal opening of the irrigation catheter 308 in different directions, which can include facing in a proximal direction. As shown in FIG. 11A, the opening into the catheter 302 can be offset at a forward plane relative to the opening into the irrigation catheter 308, which can control the plume (e.g., softening and/or emulsification zone) of the heated fluid. For example, the plume (e.g., softening and/or emulsification zone) can include a funnel and/or conde shape. The configuration of the plume can protect a wall of the blood vessel and/or other areas that are not intended to be exposed to treatment.

In some variants, the catheter 302 can be an irrigation catheter and the catheter 308 can be an aspiration catheter. For example, irrigation can be performed in the periphery and aspiration in the middle (e.g., concentric) with low pressure at the middle to reverse the flow of irrigation fluid back into the aspiration catheter to control the plume. In some variants, irrigation can be performed through peripheral apertures and aspiration can be performed centrally (e.g., at a distal open end of the aspiration catheter).

As illustrated in FIG. 11B, the irrigation catheter 308 can include one or more heated elements 310 (e.g., metal and/or metal alloy elements that can be heated). The one or more heated elements 310 can be disposed at the distal end 309 of the irrigation catheter 308, which can include surrounding the distal end 309 (e.g., open distal end 309). In some variants, fluid traveling through the irrigation catheter 308 can be heated by the one or more heated elements 310. As illustrated in FIG. 11C, a temperature sensor 312 can be disposed at the heated fluid jet 306 to sense the temperature. In some variants, the temperature of the fluid jet 306 (e.g., one or more heated elements 310) can be adjusted based on the temperature sensed by the temperature sensor 312. As illustrated in FIG. 11D, in some variants, the irrigation catheter 308 can include a closed distal end 314 and one or more lateral openings 316. The one or more lateral openings 316 can be open in a direction that is angled and/or generally perpendicular relative to the longitudinal axis of the irrigation catheter 308, which can facilitate rapid aspiration by way of the catheter 302 to localize the heated fluid jet 306. In some variants, the distal end 314 may be advanced distally of the distal end of the catheter 302. The heated fluid for the fluid jet 306 can be heated by way of indirect or direct heat with one or more energy sources, which may at least include heat, radio frequency, laser, electricity (e.g., current), resistive heating, inductive heating, ultrasound, heated fluid, nuclear, and/or others.

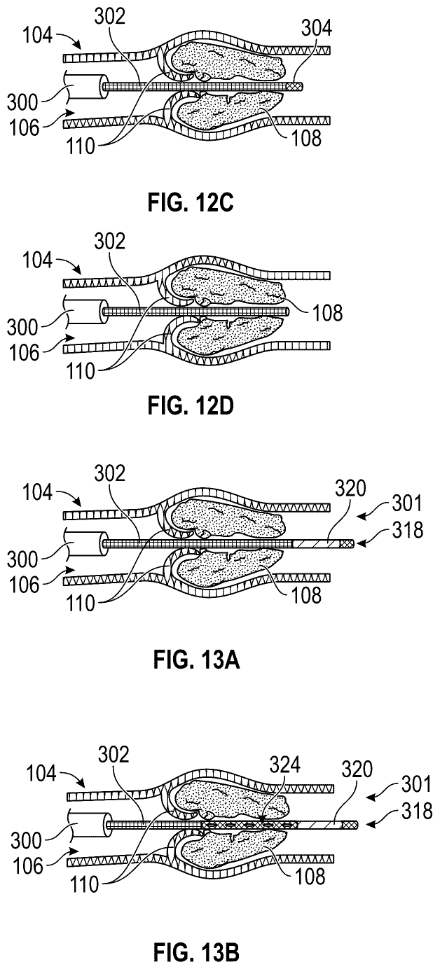

FIGS. 12A-12D illustrate a method of penetrating and/or crossing a thrombus 108 with a heated element 304 (e.g., heated member, heated wire, heated guidewire, heated tip, heated end). As detailed above in reference to FIG. 12A, the catheter 302 can be advanced to proximate the thrombus 108. As illustrated in FIG. 12B, a heated element 304 can be deployed from within the catheter 302 and advanced distally through the thrombus 108. The catheter 302 can be advanced with the heated element 304 in some variants to enable the distal end of the catheter 302 to cross over the thrombus 108, as illustrated in FIG. 12D. As described herein, the heated element 304 can be heated to at least temperatures described herein. The heated element 304 may be heated directly or indirectly with one or more energy sources, which may at least include heat, radio frequency, laser, electricity (e.g., current), resistive heating, inductive heating, ultrasound, heated fluid, nuclear, and/or others. The heat from the heated element 304 can ease penetration through the thrombus 108. The heat from the heated element 304 can soften and/or emulsify the thrombus 108, which can include emulsifying a center (e.g., core) of the thrombus 108. With the distal end of the catheter 302 distal of the thrombus 108, the heated element 304 can be retracted through the inner catheter 302, leaving the catheter 302 in place, as illustrated in FIG. 12D.

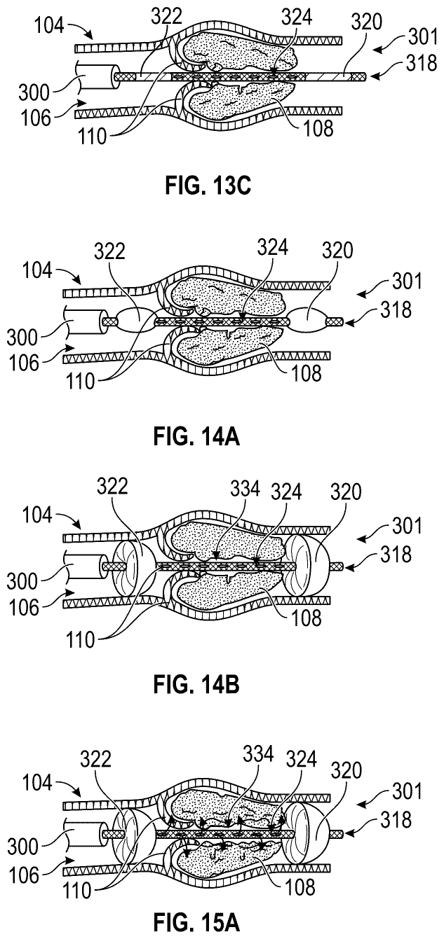

FIGS. 13A-13C illustrate a method of delivering an irrigation and aspiration catheter 318 of a thermal system 301 to an occlusion site. As illustrated in FIG. 13A, the irrigation and aspiration catheter 318 can be advanced distally of the catheter 302. As illustrated in FIGS. 13B and 13C, the catheter 302 can be retracted proximally to expose the irrigation and aspiration catheter 318. The irrigation and aspiration catheter 318 can include a distal balloon 320 and/or proximal balloon 322. The irrigation and aspiration catheter 318 can include one or more apertures 324 positioned between the distal balloon 320 and proximal balloon 322. In some variants, a distal end of the irrigation and aspiration catheter 318 can be heated and can be used to penetrate and/or cross over the thrombus 108, which can include facilitating the cross over of the catheter 302 which can replace or cooperate with the heated element 304. In some variants, a distal end of the catheter 302 can be heated and can be used to penetrate and/or cross over the thrombus 108, which can replace or cooperate with the heated element 304.

FIGS. 14A and 14B illustrate a method of expanding (e.g., inflating) the distal balloon 320 and proximal balloon 322 to contact a wall of the blood vessel. The expansion of the distal balloon 320 and proximal balloon 322 can fluidically isolate the segment of the blood vessel, e.g., vein 104, between the distal balloon 320 and proximal balloon 322. The distal balloon 320 and the proximal balloon 322 can be expanded (e.g., inflated) with a variety of techniques, which can at least include filling with fluid and/or gas. The distal balloon 320 and the proximal balloon 322 can be expanded (e.g., inflated) manually and/or automatically.

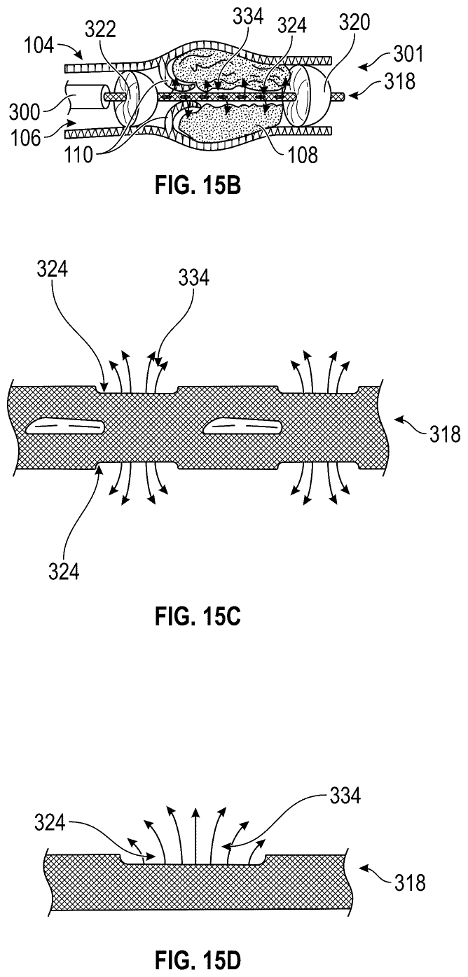

With the occlusion site (e.g., thrombus 108 and/or plaque) fluidically isolated by the distal balloon 320 and proximal balloon 322 from the remainder of the blood vessel (e.g., vein 104), heated fluid 334 can be delivered to the occlusion site by way of the apertures 324 in the irrigation and aspiration catheter 318, as illustrated in FIGS. 15A and 15B, to soften and/or emulsify the thrombus 108 and/or plaque. As illustrated in FIG. 15C, the apertures 324 can be disposed circumferentially around the irrigation and aspiration catheter 318. As illustrated in FIG. 15D, the apertures 324 can be disposed on one side of the irrigation and aspiration catheter 318. The apertures 324 can be varying shapes and/or sizes. The apertures 324 can be the same size and/or shape.

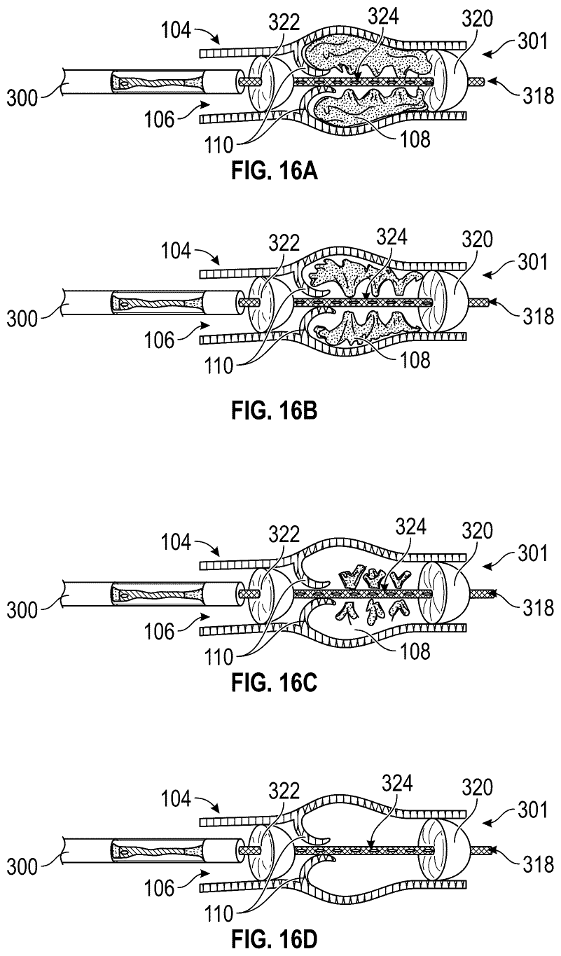

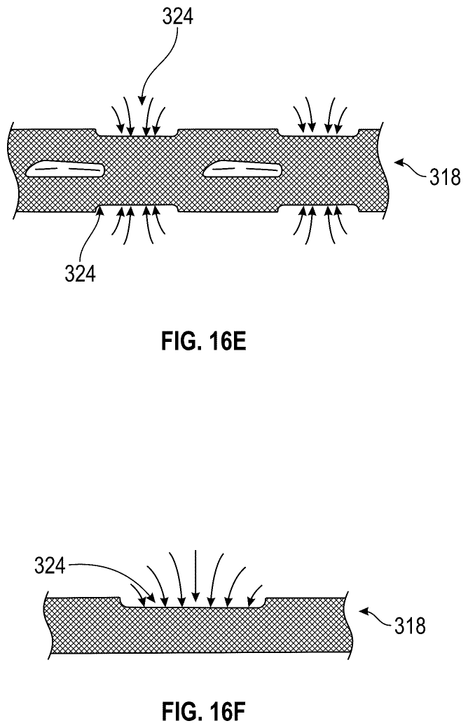

As illustrated in FIGS. 16A-16F, the softened and/or emulsified thrombus 108 and/or plaque can be aspirated by way of the apertures 324. In some variants, the irrigation and aspiration catheter 318 can cease delivering heated fluid to the occlusion site prior to aspiration. In some variants, the irrigation and aspiration catheter 318 can simultaneously deliver heated fluid to the occlusion site and aspirate the softened and/or emulsified thrombus 108 and/or plaque. In some variants, some of the apertures 324 can be used for delivering the heated fluid to the occlusion site while others can be used to aspirate the softened and/or emulsified thrombus 108 and/or plaque. In some variants, the heated fluid can be aspirated by way of the apertures 324. In some variants, the blood aspirated by the apertures 324 can be returned after filtering. The aspirated matter (e.g., heated fluid, softened and/or emulsified thrombus 108 and/or plaque, etc.) can be directed to a waste reservoir (e.g., tank, bag, bottle, container, syringe, chamber, vessel, compartment, etc.).

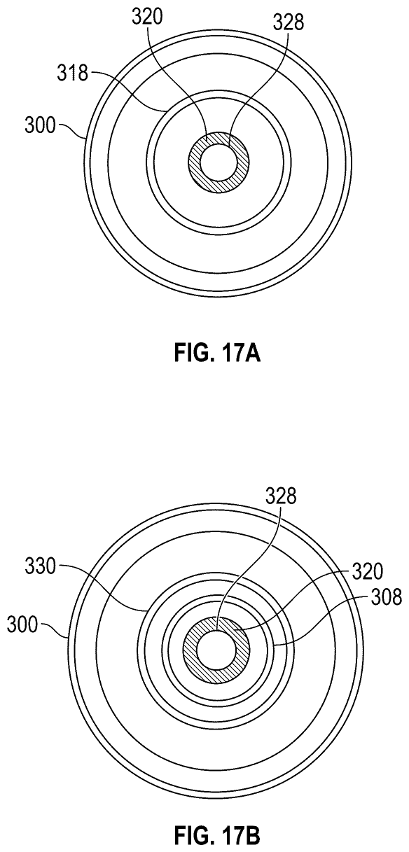

FIGS. 17A and 17B illustrate cross-sectional views of example catheter configurations to deliver heated fluid to an occlusion site and aspirate matter (e.g., heated fluid, softened and/or emulsified thrombus and/or plaque, etc.) from the occlusion site. As illustrated in FIG. 17A, the distal balloon 320 can be inflated and/or deflated with a distal balloon pneumatic lumen 328 that can deliver and/or remove gas and/or fluid from the distal balloon 320. The irrigation and aspiration catheter 318 can deliver heated fluid to the occlusion site and/or aspirate matter from the occlusion site. In some variants, the distal balloon pneumatic lumen 328 can be disposed in a catheter upon which the distal balloon 320 is disposed that can be delivered through the irrigation and aspiration catheter 318. In some variants, the proximal balloon can be disposed on the irrigation and aspiration catheter 318. As illustrated in FIG. 17B, a separate irrigation catheter 308 can be used to deliver heated fluid to an occlusion site and a separate aspiration catheter 330 can be used to aspirate matter from the occlusion site. The irrigation catheter 308 can be delivered through the aspiration catheter 330. The distal balloon 320 can be inflated and/or deflated with a distal balloon pneumatic lumen 328 that can deliver and/or remove gas and/or fluid from the distal balloon 320. In some variants, the distal balloon pneumatic lumen 328 can be disposed in a catheter upon which the distal balloon 320 is disposed that can be delivered through the irrigation catheter 308. In some variants, the proximal balloon can be disposed on the aspiration catheter 330.

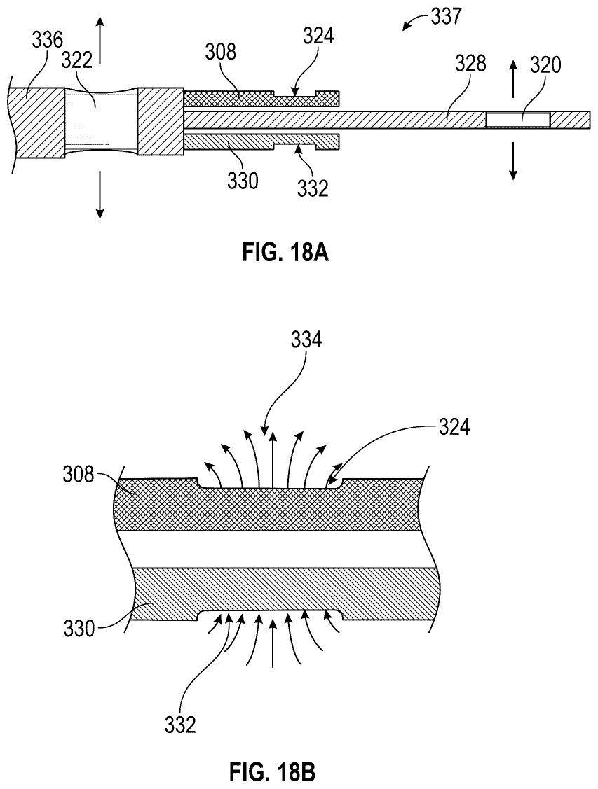

FIGS. 18A and 18B illustrate example catheters of a thermal system 337 to deliver heated fluid to an occlusion site and aspirate matter (e.g., softened and/or emulsified thrombus and/or plaque) from the occlusion site. As illustrated in FIG. 18A, the thermal system 337 can include a catheter 336 (e.g., outer catheter, working catheter). The catheter 336 can include a proximal balloon 322, which can be disposed on a distal portion thereof. A catheter 328 (e.g., inner catheter, distal-balloon catheter) can be advanced distally out of the catheter 336. The catheter 328 can include the distal balloon 320. The thermal system 337 can include an irrigation catheter 308 that can be advanced out of the catheter 336. The irrigation catheter 308 can include an aperture 324 through which heated fluid can be delivered to an occlusion site. In some variants, the irrigation catheter 308 can include a plurality of apertures 324 to deliver heated fluid. The thermal system 337 can include an aspiration catheter 330 that can be advanced out of the catheter 336. The aspiration catheter 330 can include an aperture 332 to aspirate matter (e.g., heated fluid, softened and/or emulsified thrombus and/or plaque, etc.) from an occlusion site. In some variants, the aspiration catheter 330 can include a plurality of apertures 332. In some variants, the catheter 336 can be a multi-lumen catheter, which can include a lumen for each of the irrigation catheter 308, aspiration catheter 330, and/or catheter 328. In some variants, the catheter 336 can include a single lumen for the irrigation catheter 308, aspiration catheter 330, and catheter 328. As shown in FIG. 18B, heated fluid 334 can be delivered to an occlusion site by way of the aperture 324 and matter (e.g., heated fluid, softened and/or emulsified thrombus and/or plaque, etc.) can be aspirated by way of the aperture 332. In some variants, the aperture 324 and/or aperture 332 can be elongate apertures. In some variants, the aperture 324 and/or aperture 332 can include a curved periphery. In some variants, the aperture 324 and aperture 332 can face opposite directions.

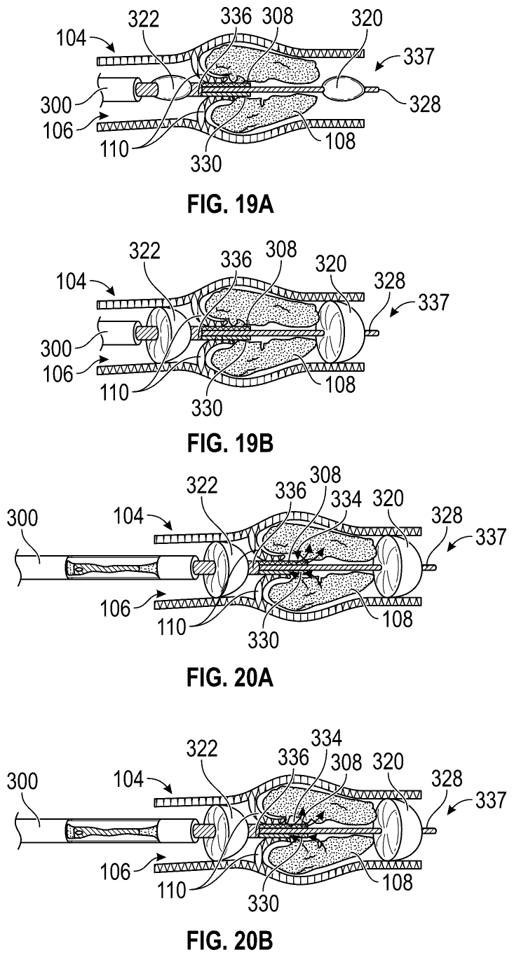

The catheters of the thermal system 337 can be positioned at an occlusion site at least using the techniques described herein. For example, a guidewire can be navigated to proximal of an occlusion site. A catheter 300 can be advanced over the guidewire to the occlusion site. An inner catheter (e.g., catheter 302) can be advanced distally out of the catheter 300. A heated element (e.g., heated element 304) can be advanced distally of the distal end of the inner catheter. The heated element and the inner catheter can be advanced together with the heated element easing penetration and crossing over of the occlusion (e.g., thrombus). With the distal end of the inner catheter distal of the occlusion (e.g., thrombus and/or plaque), the heated element can be retracted. The catheter 336, irrigation catheter 308, aspiration catheter 330, and/or catheter 328 can be advanced within the inner catheter such that the distal balloon 320 is distal of the occlusion site and the proximal balloon 322 is proximal of the occlusion site. The inner catheter can be retracted to expose the proximal balloon 322, aperture 324 of the irrigation catheter 308, aperture 332 of the aspiration catheter 330, and/or distal balloon 320. In some variants, the irrigation catheter 308 and/or aspiration catheter 330 can be advanced out of the catheter 336 to position the aperture 324 of the irrigation catheter 308 and aperture 332 of the aspiration catheter 330 at the occlusion site (e.g., at the thrombus and/or plaque). As illustrated in FIGS. 19A and 19B, the distal balloon 320 and proximal balloon 322 can be expanded (e.g., inflated) to fluidically isolate the segment of the blood vessel (e.g., vein 104) between the distal balloon 320 and proximal balloon 322 from the remainder of the blood vessel (e.g., vein 104).

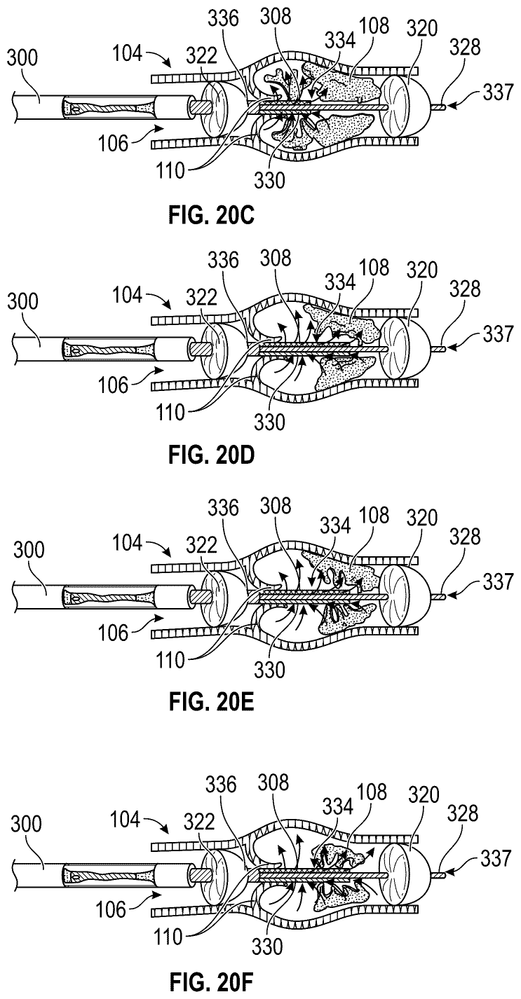

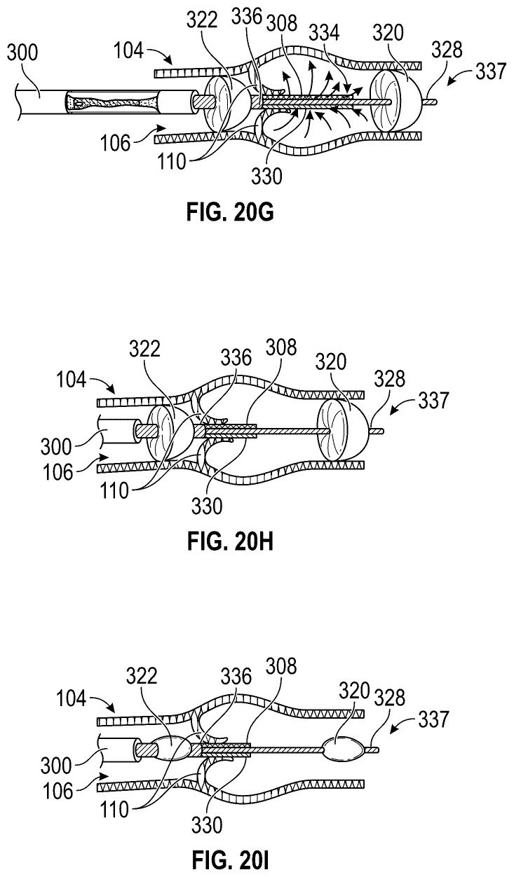

As illustrated in FIGS. 20A-20G, heated fluid 334 can be delivered to the thrombus 108 by way of the aperture 324 of the irrigation catheter 308 to soften and/or emulsify the thrombus 108. The heated fluid 334, softened and/or emulsified thrombus 108, and/or other matter can be aspirated through the aperture 332 of the aspiration catheter 330. The irrigation catheter 308 and/or aspiration catheter 330 can be advanced distally through the thrombus 108 as the heated fluid 334 softens and/or emulsifies the thrombus 108. In some variants, the irrigation catheter 308 can deliver the heated fluid 334 while the aspiration catheter 330 aspirates. In some variants, the irrigation catheter 308 can deliver the heated fluid 334 and the aspiration catheter 330 can aspirate at different times (e.g., in sequence). As shown in FIGS. 20H and 20I, the distal balloon 320 and proximal balloon 322 can be deflated for removal. Once the distal balloon 320 and the proximal balloon 322 are deflated, the catheter 336, irrigation catheter 308, aspiration catheter 330, and/or catheter 328 can be retracted proximally into the catheter 300 for removal.

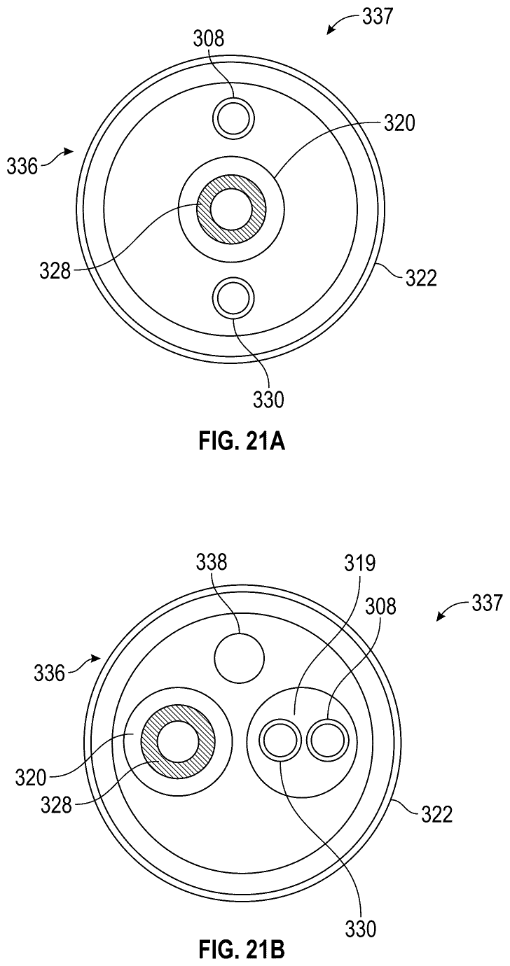

FIGS. 21A and 21B illustrate cross-sectional views of example catheter configurations to deliver heated fluid to an occlusion site and aspirate matter (e.g., heated fluid, softened and/or emulsified thrombus and/or plaque, etc.) from the occlusion site. As illustrated in FIG. 21A, the aspiration catheter 330 and the irrigation catheter 308 can be disposed on opposite sides of the catheter 328, which can include being on opposite sides of the distal balloon catheter 328. The catheter 328 can include a lumen (e.g., pneumatic lumen, fluid lumen, gas lumen) for fluid and/or gas to inflate or deflate the distal balloon 320. In some variants, the catheter 336 can include a single lumen for the irrigation catheter 308, catheter 328, and/or aspiration catheter 330. In some variants, the catheter 336 can include a plurality of lumens (e.g., three) with one lumen for each of the irrigation catheter 308, catheter 328, and/or aspiration catheter 330. The catheter 328 can be centered within the catheter 336 (e.g., the lumen for the catheter 328 can be centered within the catheter 336). The catheter 336 can include a lumen (e.g., pneumatic lumen, fluid lumen, gas lumen) to inflate or deflate the proximal balloon. As illustrated in FIG. 21B, in some variants, the pneumatic lumen 338 can include a pneumatic lumen 338, which can be a fluid lumen, to inflate or deflate the proximal balloon. In some variants, the aspiration catheter 330 and the irrigation catheter 308 can deploy from the same lumen 319 of the catheter 336. In some variants, the aspiration catheter 330 and irrigation catheter 308 can deploy from a multi-lumen catheter disposed in the lumen 319. In some variants, the catheter 328 can be disposed laterally of the lumen 319.

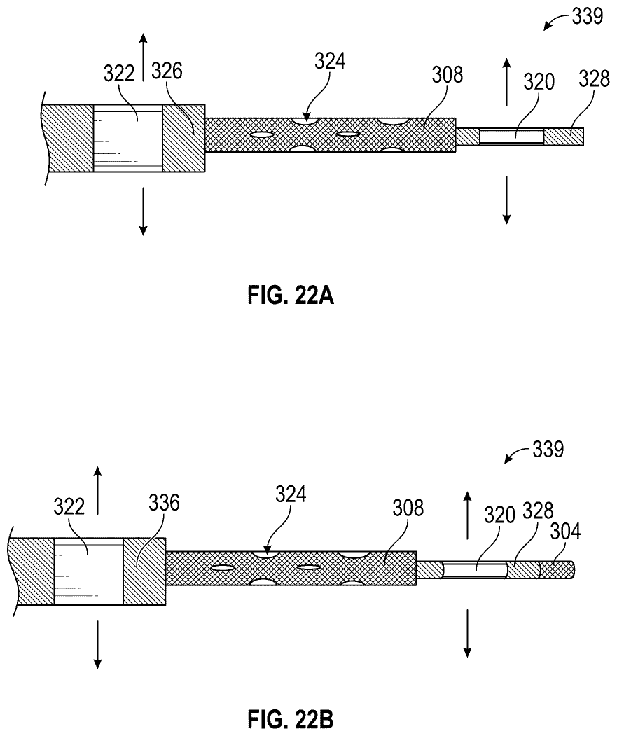

FIGS. 22A and 22B illustrate an example catheter arrangement of a thermal system 339 to deliver heated fluid to an occlusion site and aspirate material (e.g., heated fluid, softened and/or emulsified thrombus and/or plaque, etc.). The thermal system 339 can include a catheter 336 (e.g., outer catheter, working catheter). The catheter 336 can include a proximal balloon 322, which can be disposed on a distal portion thereof. The catheter 336 can aspirate material from an occlusion site. A catheter 328 (e.g., inner catheter, distal-balloon catheter) can be advanced distally out of the catheter 336. The catheter 328 can include a distal balloon 320. The thermal system 337 can include an irrigation catheter 308 that can be advanced out of the catheter 336. The irrigation catheter 308 can include one or more apertures 324 through which heated fluid can be delivered to an occlusion site. The irrigation catheter 308 can be advanced along the catheter 328 to position the one or more apertures 324 of the irrigation catheter 308 distal of the proximal balloon 322 and proximal of the distal balloon 320. As illustrated in FIG. 22B, a heated element 304 (e.g., heated wire, heated guidewire) can be deployed distally from (e.g., distally out of) the catheter 328 to ease penetration and/or crossing over an occlusion to position the distal balloon 320 distal of the occlusion site and/or position the one or more apertures 324 of the irrigation catheter 308 at the occlusion. In some variants, a distal end of the catheter 328 can include one or more heating elements to ease penetration and/or crossing over an occlusion, which can cooperate with the heated element 304 or replace the heated element 304 so that a separate heated element 304 is not used. The proximal portions of the heated element 304 can be insulated.

In use, a guidewire can be navigated to proximal of an occlusion site (e.g., site of thrombus and/or plaque). The catheter 336 and irrigation catheter 308, which may be disposed inside the catheter 336, can be advanced over the guidewire to proximal of the occlusion site. The proximal balloon 322 can be inflated to contact a surrounding wall using the techniques described herein, which can anchor the catheter 336 in place in the blood vessel (e.g., vein or artery). The guidewire can be removed. The catheter 328 can be inserted and advanced through the catheter 336. A distal end of the catheter 328 can be advanced distal of a distal end of the catheter 336 and/or a distal end of the irrigation catheter 308. A heated element 304 can be advanced distally out of the catheter 328. The heated element 304 can be heated using at least the techniques described herein. The heated element 304 can be advanced through the occlusion to crossover the occlusion. The catheter 328 can be advanced with the heated element 304 to position the distal balloon 320 distal of the occlusion. The heat from the heated element 304 can ease penetration through the occlusion, which can include softening and/or emulsifying a core (e.g., central portion) of the occlusion. The distal balloon 320 can be inflated using at least the techniques described herein. The inflated distal balloon 320 and proximal balloon 322 can isolate (e.g., fluidically isolate) the segment of the blood vessel between the distal balloon 320 and proximal balloon 322. Heated fluid can be delivered to the occlusion site by way of the apertures 324 of the irrigation catheter 308. The heated fluid can be heated to at least the temperatures described herein. As described herein, the heated fluid can be heated in a reservoir, during transport from the reservoir to the occlusion site, and/or at the occlusion site (e.g., at the apertures 324). The heated fluid can soften and/or emulsify the occlusion. The catheter 336 can aspirate the softened and/or emulsified occlusion, heated fluid, and/or other material. In some variants, aspirated blood can be filtered and returned to the blood vessel. In some variants, the guidewire can be heated to facilitate penetration and/or crossover of the occlusion, which can replace or cooperate with the heated element 304. In some variants, the catheter 328 can include a heated element at a distal end thereof that can facilitate penetration and/or crossover of the occlusion, which can replace or cooperate with the heated element 304.

The features of the thermal system 339 can be various sizes. For example, the proximal balloon 322 can include an outside diameter of twenty millimeters when inflated. The distal balloon catheter 328 can have an inside diameter (e.g., internal lumen diameter) of 1.66 millimeters (e.g., 5 Fr). The heated element 304 can have an outside diameter of 1.5 millimeters. The distal balloon 320 can include an outside diameter of twenty millimeters when inflated. The foregoing dimensions are exemplary and should not be considered limiting.

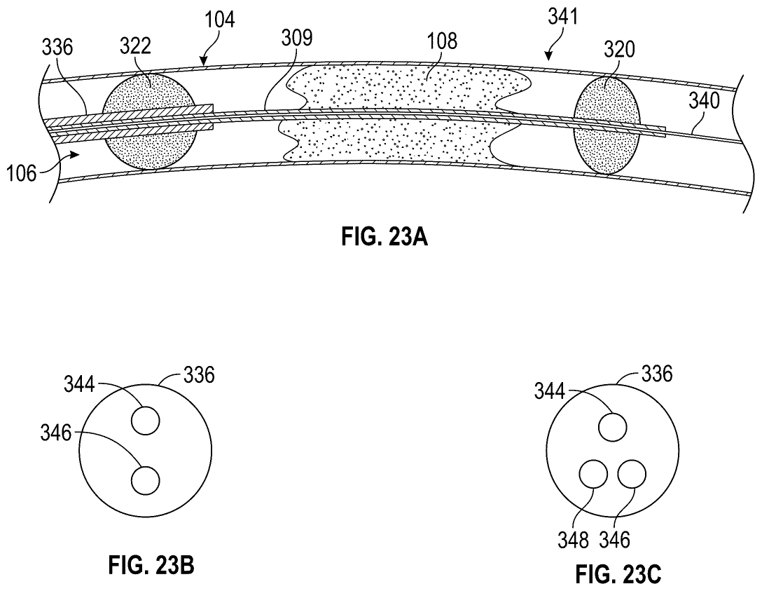

FIG. 23A illustrates catheters of a thermal system 341 positioned to deliver heated fluid to a thrombus 108 for softening and/or emulsification to facilitate aspiration. As illustrated, a guidewire 340 can be routed to a thrombus 108. In some variants, the guidewire 340 can be heated, which can include heating a distal end of the guidewire 340. The guidewire 340 can penetrate and crossover the thrombus 108. The thermal system 341 can include a catheter 336 with a proximal balloon 322. The thermal system 341 can include a distal balloon catheter 309 with a distal balloon 320. The distal balloon catheter 309 can be deployed from within the catheter 336. The catheter 336 and distal balloon catheter 309 can be advanced over the guidewire 340 to proximate the thrombus 108. The proximal balloon 322 can be inflated using at least the techniques described herein. The distal balloon catheter 309 can be advanced over the guidewire 340 to position the distal balloon 320 distal of the thrombus 108. The distal balloon 320 can be inflated using at least the techniques described herein. With the proximal balloon 322 and distal balloon 320 inflated, the segment of the vein 104 between the proximal balloon 322 and distal balloon 320 can be isolated (e.g., fluidically) from the remainder of the vein 104. Heated fluid can be delivered to the thrombus 108 by way of the catheter 336 (e.g., one or more lumens of the catheter 336). The heated fluid can soften and/or emulsify the thrombus 108. The softened and/or emulsified thrombus 108, heated fluid, and/or other material can be aspirated through the catheter 336 (e.g., one or more lumens of the catheter 336) for removal. In some variants, the aspirated blood can be filtered and returned to the vein 104. In some variants, the catheter 336 can be a multi-lumen catheter (e.g., include multiple lumens). For example, as illustrated in the cross-section shown in FIG. 23B, the catheter 336 can include two lumens, which can include an irrigation lumen 344 to deliver heated fluid to the thrombus 108 and an aspiration lumen 346 to aspirate softened and/or emulsified thrombus 108, heated fluid, and/or other material. The irrigation lumen 344 can be a working lumen through which the distal balloon catheter 309 can be deployed and/or guidewire 340. As illustrated in the cross-section shown in FIG. 23C, the catheter 336 can include three lumens, which can include a irrigation lumen 344, an aspiration lumen 346, and/or a working lumen 348 through which the distal balloon catheter 309 and/or guidewire 340 can be deployed. The distal balloon catheter 309 can include apertures to deliver the heated fluid. In some variants, a separate irrigation catheter can be advanced through the irrigation lumen 344 to deliver the heated fluid.

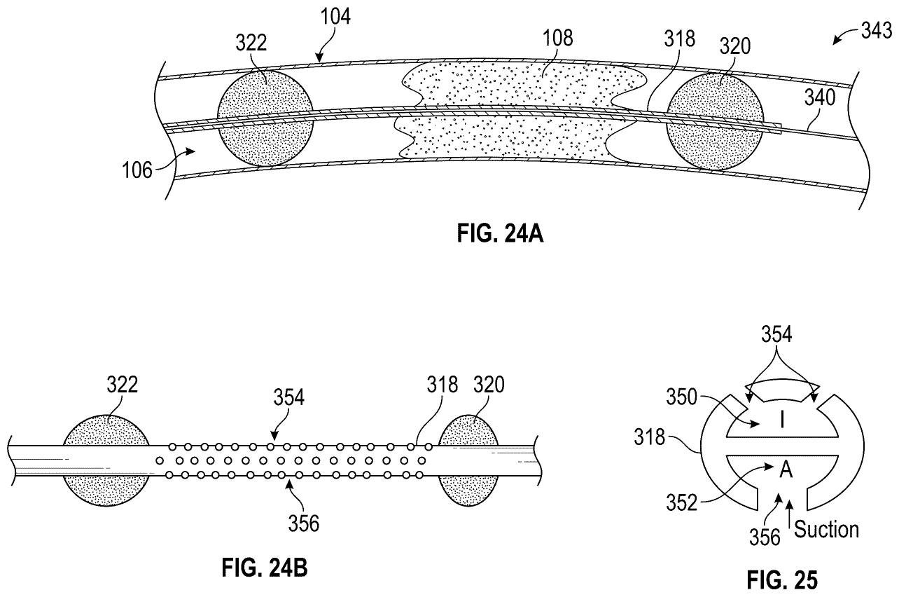

FIG. 24A illustrates an irrigation and aspiration catheter 318 of a thermal system 343 positioned to deliver heated fluid to a thrombus 108 for softening and/or emulsification to facilitate aspiration. As illustrated, the irrigation and aspiration catheter 318 can include a distal balloon 320 and a proximal balloon 322. A guidewire 340 can be navigated to a thrombus 108. The guidewire 340 can penetrate and/or crossover the thrombus 108. The guidewire 340, in some variants, can be heated, which can ease penetration and/or crossover. The irrigation and aspiration catheter 318 can be advanced over the guidewire 340 to position the distal balloon 320 distal of the thrombus 108 and the proximal balloon 322 proximal of the thrombus. The distal balloon 320 and proximal balloon 322 can be inflated, which can isolate (e.g., fluidically isolate) the segment of the vein 104 between the distal balloon 320 and proximal balloon 322. As illustrated in FIG. 24B, heated fluid can be delivered to the thrombus 108 by way of one or more apertures 354 of the irrigation and aspiration catheter 318. The heated fluid can soften and/or emulsify the thrombus 108. The softened and/or emulsified thrombus 108 can be aspirated by way of one or more apertures 356 of the irrigation and aspiration catheter 318, as illustrated in FIG. 24B. The irrigation and aspiration catheter 318 can, in some variants, irrigate and aspirate simultaneously or in sequence (e.g., stages). As illustrated in the cross-section shown in FIG. 25, the irrigation and aspiration catheter 318 can include an irrigation lumen 350 to deliver heated fluid and/or an aspiration lumen 352 to facilitate aspiration.

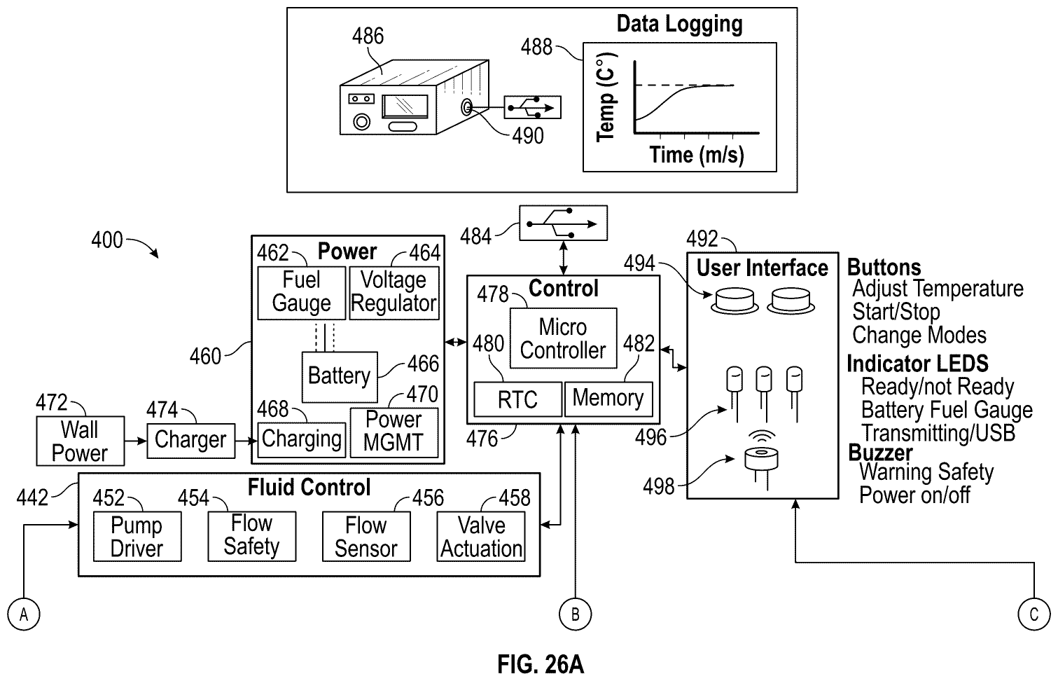

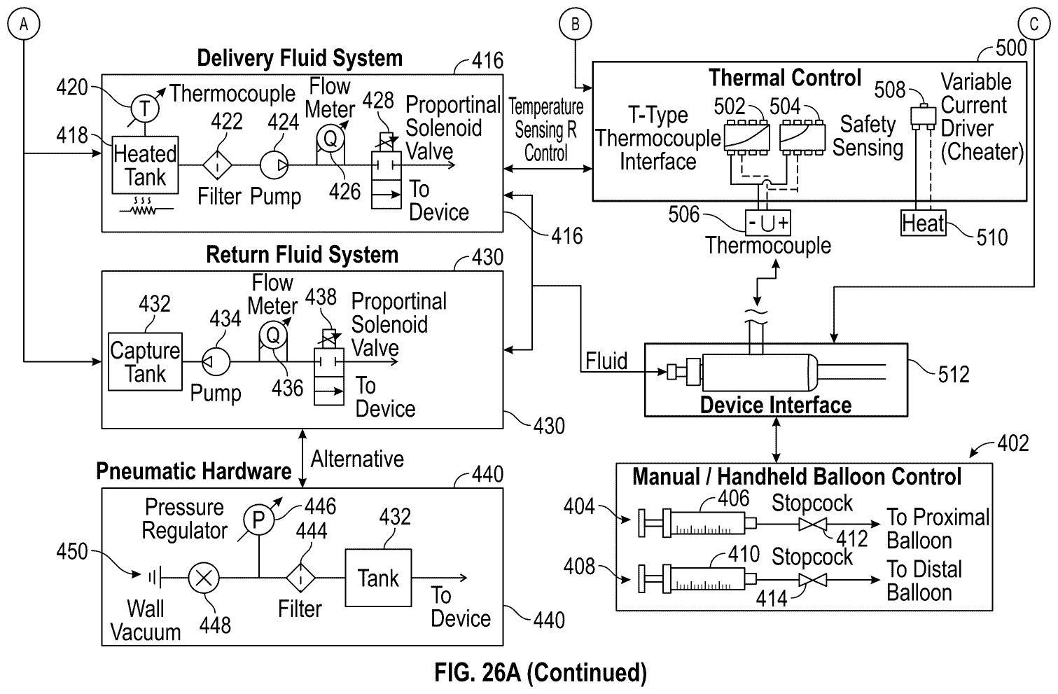

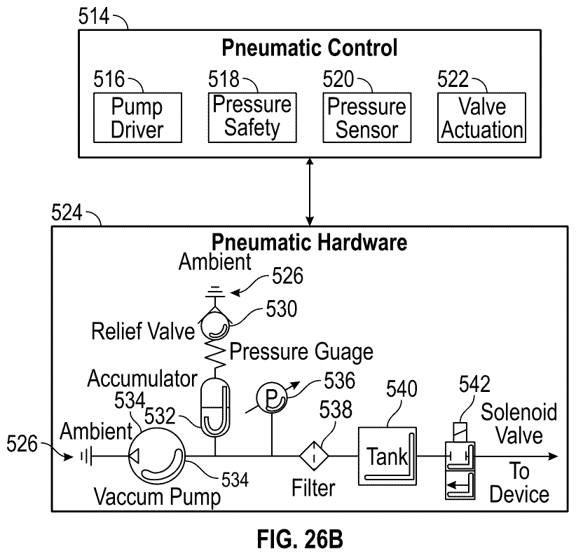

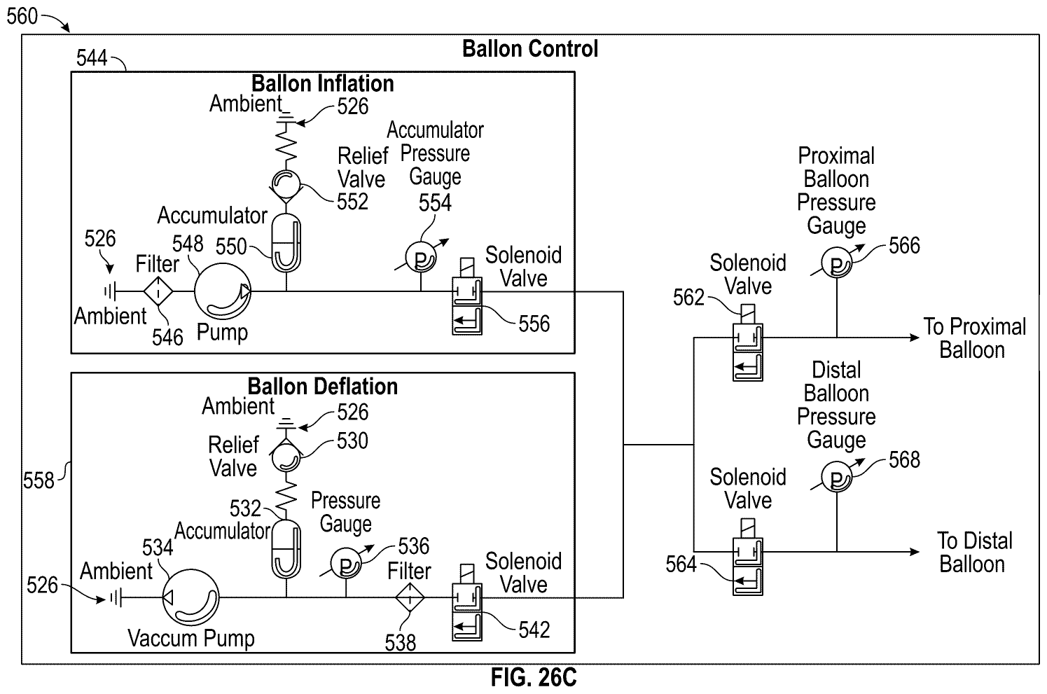

FIG. 26A illustrates a schematic of a thermal system 400 (e.g., thermal thrombectomy system, thermal atherectomy system, thrombectomy system, atherectomy system). The thermal system 400 can be used to emulsify and/or aspirate a thrombus, plaque, and/or other occlusive material from a blood vessel, such as a vein and/or artery. The thermal system 400 can include more or less features than illustrated. The thermal system 400 can include any of the features described in relation to other systems, devices, catheters, and/or methods described herein.

The thermal system 400 can include a thermal control system 500 (e.g., temperature control system). The thermal control system 500 can control the temperature (e.g., heating) of the heating element to penetrate and crossover an occlusion and/or the heated fluid, which can be used to penetrate, soften, and/or emulsify an occlusion. The thermal control system 500 can control the temperature (e.g., heat) other heating elements and/or guidewires described herein.

The thermal control system 500 can include one or more heaters 508 (e.g., variable current driver, current driver) that can heat one or more heating elements 510, which can include heating by way of electricity and/or the other techniques described herein. The one or more heaters 508 can heat the one or more heating elements 510 to at least temperatures described herein or above to accommodate for heat loss. The one or more heating elements 510 can heat the heated fluid and/or include the heated element 304 and/or other heated elements described herein. Wiring can be routed through a device interface 512 to the heated element 304 that is disposed inside the blood vessel of the patient. A heating element 510 can heat fluid in a tank 418 (e.g., reservoir, bag, bottle, container, syringe, chamber, vessel, compartment, etc.) of a fluid delivery system 416 for delivery. In some variants, a heating element 510 can heat fluid in a catheter and/or at a distal end of a catheter.

The thermal control system 500 can include a temperature sensor interface 502 (e.g., thermocouple interface, T-type thermocouple interface). The temperature sensor interface 502 can interface with one or more temperature sensors 506 (e.g., thermocouple(s)). The one or more temperature sensor 506 can sense the temperature of the heated fluid in the tank 418, in the catheter(s), and/or at the distal end(s) of the catheter(s). Wiring for a temperature sensor 506 can be routed through a device interface 512 to within a catheter and/or at the distal end of the catheter. The thermal control system 500 can modulate the temperature of the one or more heating elements 510 based on temperatures sensed by the one or more temperature sensors 506. The thermal control system 500 can include a safety sensing unit 504. The safety sensing unit 504 can, if certain temperatures are detected by the one or more temperature sensors 506, initiate a safety protocol, which can at least include cease applying heat by way of the heater 508, cease pumping fluid to the occlusion site, cease aspirating matter from the occlusion site, close one or more valves, open one or more valves, etc.

The thermal system 400 can include a fluid delivery system 416. The fluid delivery system 416 can deliver fluid to an occlusion by way of a catheter. The fluid delivery system 416 can include a tank 418 (e.g. reservoir, bag, bottle, container, syringe, chamber, vessel, compartment, etc.) that can hold a fluid. In some variants, the fluid can be heated in the tank 418. For example, one or more heating elements 510 of the thermal control system 500 can be disposed at the tank 418 to heat the fluid therein. The fluid in the tank 418 can be heated to the temperatures described herein or higher to account for heat loss. A temperature sensor 420 (e.g., thermocouple) can be disposed at the tank 418 to measure a temperature of the fluid. The temperature sensed by the temperature sensor 420 can be communicated to the thermal control system 500 to modulate the temperature of the fluid based on the measured temperature. The fluid delivery system 416 can include a pump 424 to move fluid from the tank 418 to an occlusion site by way of a catheter, which can include passing through the device interface 512. The fluid delivery system 416 can include a flow meter 426 to sense the flow rate of the fluid. The fluid delivery system 416 can include a filter 422 that can filter the fluid prior to delivery at the occlusion site. The fluid delivery system 416 can include a valve 428 (e.g., solenoid valve, proportional solenoid valve) that can open and close.

The thermal system 400 can include a fluid return system 430 (e.g., aspiration system). The fluid return system 430 can aspirate softened and/or emulsified thrombus and/or plaque, heated fluid, and/or other material from an occlusion site in a blood vessel. The fluid return system 430 can include a tank 432 (e.g. reservoir, bag, bottle, container, syringe, chamber, vessel, compartment, etc.) that can hold matter aspirated from the blood vessel (e.g., softened and/or emulsified thrombus and/or plaque, heated fluid, etc.). The thermal system 400 can include a pump 434 can move (e.g., suck, aspirate, vacuum, extract, suction, remove) matter from the blood vessel through a catheter into the tank 432. The fluid return system 430 can include a flow meter 436 that can sense the flow rate of the matter aspirated from the blood vessel as the matter is pumped to the tank 432. The fluid return system 430 can include a valve 438 (e.g., solenoid valve, proportional solenoid valve) that can open and close.

The thermal system 400 can include a fluid control system 442 (e.g., fluid delivery and aspiration control system). The fluid control system 442 can include one or more pump drivers 452. The pump driver(s) 452 can drive the pump 424 and/or pump 434. The fluid control system 442 can include one or more flow sensor(s) 456 that can sense the flow rates in the thermal system 400. The pump driver(s) 452 can make adjustments based on sensed flow rates. The fluid control system 442 can include one or more valve actuation units 458 that can acuate the valves in the thermal system 400, which can include the valve 428 and/or valve 438. The fluid control system 442 can include a flow safety unit 454. When the one or more flow sensors 456 sense certain flow rates, the flow safety unit(s) 454 can initiate one or more safety protocols (e.g., cease driving the pump 424 and/or pump 434 with the one or more pump drivers 452, close the valve 428 and/or valve 438, etc.).

In some variants, the fluid return system 430 can be replaced by a pneumatic system 440. The pneumatic system 440 can aspirate softened and/or emulsified thrombus and/or plaque, heated fluid, and/or other material from an occlusion site in a blood vessel. The pneumatic system 440 can include the tank 432 to hold matter aspirated from the blood vessel. The pneumatic system 440 can be coupled to a vacuum source 450, which can include a wall vacuum source. The pneumatic system 440 can include a filter 444. The filter 444 can filter unwanted matter from flowing to the vacuum source 450. The pneumatic system 440 can include a pressure regulator 446 that can regulate the pressure in the pneumatic system 440. The pneumatic system 440 can include a light source 448, such as a bulb, LED, etc.

The thermal system 400 can include a manual balloon control system 402 (e.g., balloon control system). The manual balloon control system 402 can include a proximal balloon control system 404 and/or a distal balloon control system 408. The proximal balloon control system 404 can inflate and/or deflate the proximal balloon. The distal balloon control system 408 can inflate and/or deflate the distal balloon. The proximal balloon control system 404 can include a syringe 406 that can be filled with a fluid and/or gas that can be urged into the proximal balloon for inflation and urged back into the syringe 406 for deflation. The proximal balloon control system 404 can include a valve 412 (e.g., stopcock) that can open or close to prevent or allow movement of fluid and/or gas by the syringe 406. The distal balloon control system 408 can include a syringe 410 that can be filled with a fluid and/or gas that can be urged into the distal balloon for inflation and urged back into the syringe 410 for deflation. The distal balloon control system 408 can include a valve 414 (e.g., stopcock) that can open or close to prevent or allow movement of fluid and/or gas by the syringe 410.

The thermal system 400 can include a power system 460. The power system 460 can include a battery 466, which can be rechargeable. The power system 400 can include a gauge 462. The gauge 462 can indicate the status of the battery 466 (e.g., percentage charged, etc.). The power system 460 can include a charging interface 468. The charging interface 468 can interface with a cable 474 (e.g., charging cable) that can interface with a power source 472 (e.g., outlet) to charge the battery 466 and/or directly power the system 400. The power system 460 can include a power management unit 470 and/or voltage regulator 464.

The system 400 can include a control system 476. The control system 476 can execute the methods described herein. The control system 476 can include a controller 478 (e.g., micro-controller), real-time clock 480 (e.g., RTC), and/or memory 482. The real-time clock 480 can be used to monitor durations of methods and/or steps of methods described herein (e.g., duration heating element is at a temperature, duration of heated fluid delivery, duration of aspiration, etc.). The real-time clock 480 can be used to identify triggering events for the system 400 to respond to. The system 400 can include a communication interface 484 (e.g., USB port) that can facilitate connecting the system 400 to a computing system to communicate data. For example, the thermal system 400 can be communicatively coupled with a computing device 486. A data cable can interface with the communication interface 484 of the thermal system 400 and a communication interface 490 (e.g., USB port) of the computing device 486 to communicate data. The computing device 486 can log data from the thermal system 400. The computing device 486 can analyze data from the thermal system 400, which can include producing a data log 488 (e.g., one or more graphs) based on data from the thermal system 400.

The system 400 can include a user interface system 492. The user interface system 492 can include one or more button(s) 494, which can at least be used to adjust temperature of the heating element and/or heated fluid, start/stop heating, delivery of heated fluid, and/or aspiration, change modes, adjust flow rates and/or pressure, adjust aperture sizes, open and/or shut valves, power on/off, and/or other adjustments. The user interface system 492 can, in some variants, include dial(s), switch(es), display(s), touchpad(s), touchscreen(s), knob(s), trigger(s), indicator(s), gauge(s), slider(s), and/or other features. The user interface system 492 can include indicator(s) 496 (e.g., indicator lights, such as LEDs). The indicator(s) 496 can visually indicate when the system 400 is ready for operation and/or not ready for operation. The indicator(s) 496 can visually indicate when the system 400 is aspirating, delivering heated fluid, and/or heating a heating element. The indicator(s) 496 can indicate the charge level of the battery 466. The indicator(s) 496 can indicate when the system 400 is communicating with another computing system (e.g., transmitting data). The indicator(s) 496 can emit warnings. The indicator(s) 496 can emit various colors and/or patterns of light. The user interface system 492 can include a speaker(s) 498 (e.g., buzzer(s)), which can emit audible sounds to communicate warnings, alert of safety issues, and/or emit sounds when various actions are performed (e.g., power on/off). In some variants, the user interface system 492 can include one or more displays, touchscreens, microphones for spoken commands, etc. In some variants, the indicator(s) 496, speaker(s) 498, display, touchscreen, etc. can indicate the status of the system 400, which can at least include indicating when the heated fluid is being delivered to an occlusion site, how long the heated fluid has been at a temperature or above a temperature, a temperature of the heated fluid, when the system 400 is aspirating, etc.

The system 400, in some variants, can include a wired communication interface and/or a wireless communication interface to communicate with other computing devices. The system 318 can include a processor and/or other hardware to perform the methods described herein.

In some variants, the thermal system 400 can include the pneumatic system 524 (e.g., aspiration system) to facilitate aspiration, as illustrated in FIG. 26B. The pneumatic system 524 can be used to aspirate heated fluid, thrombus, plaque, and/or other occlusion from the blood vessel of the patient. The pneumatic system 524 can include a pump 534 (e.g., vacuum pump) with access to the ambient environment 528, accumulator 532, relief valve 530 with access to the ambient environment 528, pressure gauge 536, filter 538, tank 540 (e.g. reservoir, bag, bottle, container, syringe, chamber, vessel, compartment, etc.), and/or valve 542 (e.g., solenoid valve). The tank 540 can receive aspirated material (e.g., thrombi and/or other occlusive material). The system 400 can include a gauge to indicate the fill level of the tank 540, which can include a gauge of the user interface 492. The pneumatic system 524 can be operatively coupled to the device interface 5121 to facilitate aspiration.

The system 400 can include a pneumatic control system 514 (e.g., aspiration control system), as illustrated in FIG. 26B. The pneumatic control system 514 can include a pump driver 516. The pump driver 516 can drive the vacuum pump 534. The pneumatic control system 514 can include a pressure sensor 520, which can sense pressures within the thermal system 400 and/or the blood vessel. The pneumatic control system 514 can include a valve actuation unit 522 that can acuate (e.g., open, close) the solenoid valve 542. The pneumatic control system 514 can include a pressure safety unit 518 that can monitor pressures within the thermal system 400 and/or the blood vessel. The pressure safety unit 518 can, when certain pressures are detected, initiate a safety protocol (e.g., cease driving the vacuum pump 534 with the pump driver 516).

As illustrated in FIG. 26C, the thermal system 400 can include a balloon control system 560. The balloon control system 560 can inflate and/or deflate the distal and/or proximal balloons, which can include urging gas and/or fluid into the distal and/or proximal balloons for inflation and/or urging gas and/or fluid out of the distal and/or proximal balloons for deflation. The balloon control system 560 can include a balloon inflation system 544 and/or a balloon deflation system 558. The balloon inflation system 544 can inflate the distal and/or proximal balloons. The balloon deflation system 558 can deflate the distal and/or proximal balloons.

The balloon inflation system 544 can include a pump 548, accumulator 550, relief valve 552 with access to the ambient environment 526, accumulator pressure gauge 554, filter 546, and/or valve 542 (e.g., solenoid valve). The balloon inflation system 544 can urge fluid and/or gas to the proximal balloon and/or distal balloon for inflation. The balloon deflation system 558 can include a pump 534 (e.g., vacuum pump) with access to the ambient environment 526, accumulator 532, relief valve 530 with access to the ambient environment 528, pressure gauge 536, filter 538, and/or valve 542 (e.g., solenoid valve). The balloon deflation system 558 can urge fluid and/or gas out of the proximal balloon and/or distal balloon for deflation.

A valve 562 (e.g., solenoid valve) that can open or close can be disposed proximal of the proximal balloon. With the valve 562 closed, gas and/or fluid cannot be urged into or out of the proximal balloon. A valve 564 (e.g., solenoid valve) that can open or close can be disposed proximal of the distal balloon. With the valve 564 closed, gas and/or fluid cannot be urged into or out of the distal balloon. The thermal system 400 can include a pressure gauge 566 that can sense a pressure in the proximal balloon. The thermal system 400 can include a pressure gauge 568 that can sense a pressure in the distal balloon. The valve 562 can be opened and the valve 564 closed to inflate and/or deflate the proximal balloon. The valve 564 can be opened and the valve 562 closed to inflate and/or deflate the distal balloon.

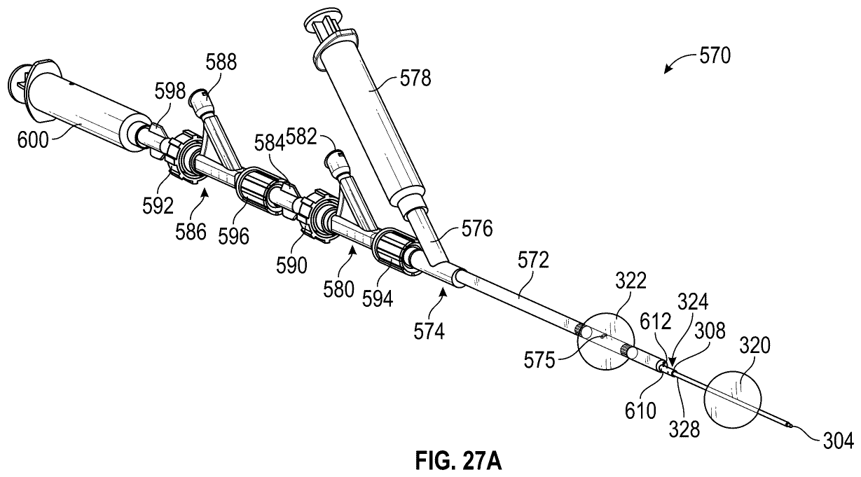

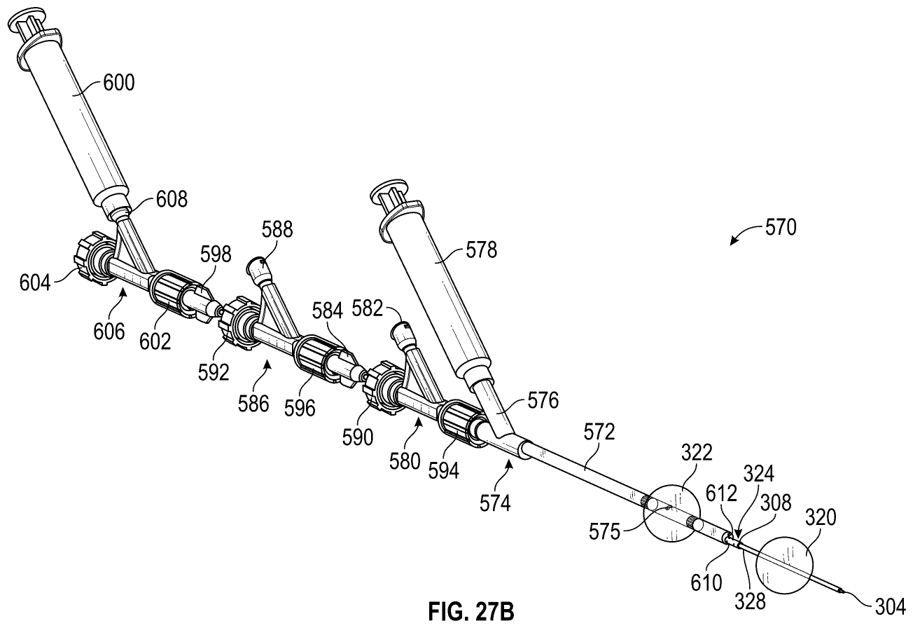

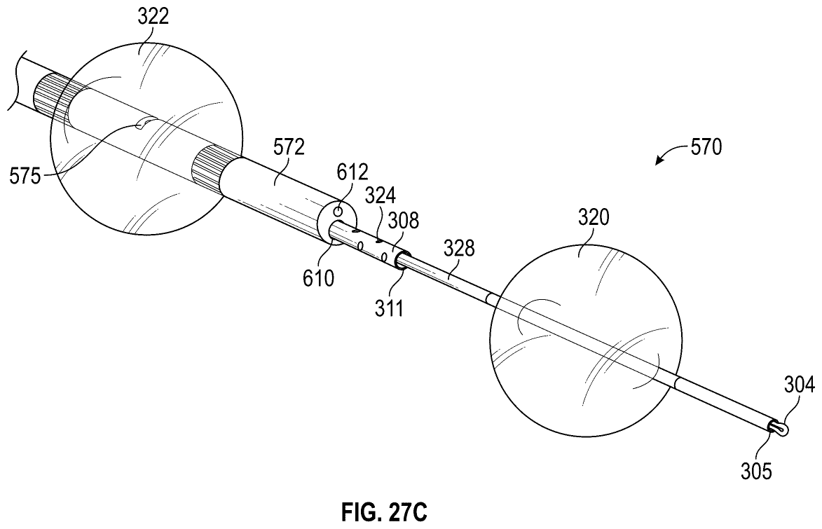

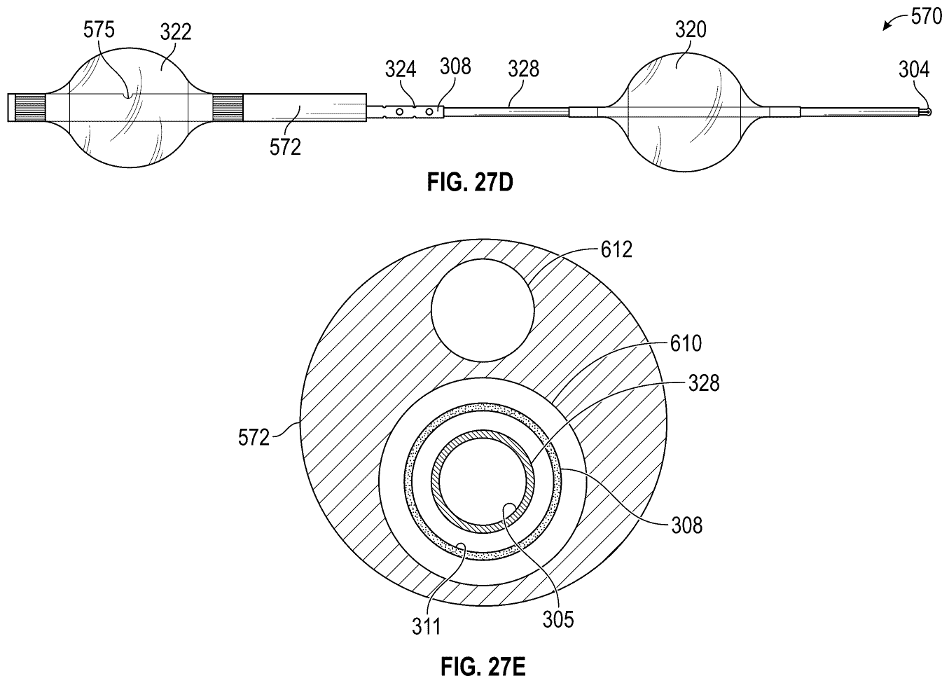

FIGS. 27A and 27B illustrate catheters of a thermal system 570 that deliver heated fluid to an occlusion to soften and/or emulsify the occlusion for aspiration. The thermal system 570 can include a multi-lumen tube 572 (e.g., multi-lumen catheter, catheter, extrusion). The multi-lumen tube 572 can include a proximal balloon 322. The proximal balloon 322 can be disposed on a distal portion (e.g., end) of the multi-lumen tube 572. The multi-lumen tube 572 can include one or more apertures 575 through which gas and/or fluid can be delivered to the proximal balloon 322 for inflation and/or removed for deflation. The multi-lumen tube 572 can include a proximal balloon lumen 612, which can be plugged at a distal end. The multi-lumen tube 572 can be fluidically coupled with the aperture 575. The multi-lumen tube 572 can be coupled to a Y connector 574 (e.g., hub, splitting hub). The Y connector 574 can include a port 576 through which gas and/or fluid can be introduced to inflate and/or removed to deflate the proximal balloon 322. A syringe 578 can be coupled to the port 576. The syringe 578 can hold gas and/or fluid that can be urged through the port 576 to the proximal balloon 322 and/or urged from the proximal balloon 322 out of the port 576. The proximal balloon lumen 612 can guide fluid and/or gas introduced through the port 576 of the Y connector 574 to the aperture 575 of the multi-lumen tube 572 to inflate the proximal balloon 322. The proximal balloon lumen 612 can guide fluid and/or gas removed from the proximal balloon 322 through the aperture 575 back to the syringe 578 by way of the port 576 of the Y connector 574. The Y connector 574 can fluidically couple the proximal balloon lumen 612 to the port 576. The Y connector 574 can be fluidically coupled with the aspiration lumen 610.

The thermal system 400 can include a Y connector 580. The Y connector 580 can include a valve, such as a hemostasis valve, to impede back flow. The Y connector 580 can be coupled to the Y connector 574 by way of a connector 594. The Y connector 580 can include an aspiration port 582. The aspiration port 582 can be fluidically coupled with an aspiration lumen 610 of the multi-lumen tube 572, which can also be a working lumen through which other devices are navigated. A pump (e.g., vacuum pump) can be coupled to the aspiration port 582 to aspirate matter from a blood vessel through the aspiration lumen 610 of the multi-lumen tube 572, which can include aspirating into a reservoir.

An irrigation catheter 308 can be disposed through the aspiration lumen 610 of the multi-lumen tube 572. The irrigation catheter 308 can be advanced distally out of the aspiration lumen 610. The irrigation catheter 308 can include one or more apertures 324 through which heated fluid can be delivered to an occlusion (e.g., thrombus and/or plaque). The one or more apertures 324 can be disposed on a distal portion (e.g., end) of the irrigation catheter 308. The irrigation catheter 308 can include an irrigation catheter hub 584. The irrigation catheter hub 584 can be coupled to a Y connector 586. A connector 596 can couple the irrigation catheter hub 584 to the Y connector 586. The Y connector 586 can include a valve, such as a hemostasis valve, to impede back flow. The Y connector 586 can include an irrigation port 588 through which heated fluid can be introduced (e.g., provided by a heated fluid delivery system). The heated fluid can travel through the irrigation port 588 of the Y connector 586, through the irrigation catheter 308, and out the one or more apertures 324 to an occlusion to soften and/or emulsify the occlusion. The heated fluid can be pumped into the irrigation port 588 and urged out the apertures 324 of the irrigation catheter 308. The thermal system 570 can include a linear actuator 590 (e.g., Tuohy Borst) to facilitate linear movement of the irrigation catheter 308. The irrigation catheter 308 can be disposed through the connector 596, linear actuator 590, Y connector 580, connector 594, Y connector 574, and/or through the aspiration lumen 610 of the multi-lumen tube 572.

A distal balloon catheter 328 can be disposed through the aspiration lumen 610 of the multi-lumen tube 572. The distal balloon catheter 328 can be disposed through the irrigation catheter 308. The distal balloon catheter 328 can be advanced distally out of the aspiration lumen 610 and/or irrigation catheter 308. The distal balloon catheter 328 can include a distal balloon 320. The distal balloon catheter 328 can be disposed on a distal portion (e.g., end) of the distal balloon catheter 328. The distal balloon catheter 328 can include one or more apertures through which gas and/or fluid can be delivered to the distal balloon 320 for inflation and/or removed for deflation. A heated element 304 (e.g., wire, looped wire) can be disposed in the distal balloon catheter 328. The heated element 304 can be heated to at least the temperatures disclosed herein by way of at least the techniques described herein. The heated element 304 can be advanced distally out of the distal balloon catheter 328. The heated element 304 can be used to penetrate and/or crossover an occlusion with the distal balloon catheter 328 to position the distal balloon 320 distal of the occlusion. The heat from the heated element 304 can ease penetration and/or crossover. The distal balloon catheter 328 can include a distal balloon catheter hub 598. The distal balloon catheter hub 598 can be coupled to a syringe 600. The syringe 600 can hold gas and/or fluid that can be urged through the distal balloon catheter 328 to the distal balloon 320 for inflation and/or urged from the distal balloon 320 through the distal balloon catheter 328 for deflation. The syringe 600 can be coaxially aligned with the distal balloon catheter hub 598. The thermal system 570 can include a linear actuator 592 (e.g., Tuohy Borst) to facilitate linear movement of the distal balloon catheter 328. The distal balloon catheter 328 can be disposed through the linear actuator 592, Y connector 586, connector 596, irrigation catheter 308, linear actuator 590, Y connector 580, connector 594, Y connector 574, and/or aspiration lumen 610 of the multi-lumen tube 572. In some variants, reservoirs (e.g., tanks, bags, bottles, containers, chambers, vessels, compartments, etc.) other than syringes can be used to hold gas and/or fluid to inflate and/or deflate the proximal balloon 322 and/or distal balloon 320. The reservoirs can be fluidically coupled with pumps to move the gas and/or fluid for inflation and/or deflation of the proximal balloon 322 and/or distal balloon 320. As illustrated in FIG. 27B, the thermal system 570 can include a Y connector 606 with a port 608. The syringe 600 can be coupled to the port 608. The Y connector 606 can be coupled to the distal balloon catheter 328. For example, the Y connector 606 can be coupled to the distal balloon catheter hub 598 of the distal balloon catheter 328. A connector 602 can couple the Y connector 606 to the distal balloon catheter hub 598. The Y connector 606, in some variants, can include a valve (e.g., hemostasis valve). The gas and/or fluid held in the syringe 600 can be urged through the port 608 of the Y connector 606 and into the distal balloon catheter 328 to inflate the distal balloon 320. The thermal system 570 can include a linear actuator 604 (e.g., Tuohy Borst) that can be disposed proximal of the Y connector 606. In some variants, the linear actuator 604 can be used to move the heated element 304 within the distal balloon catheter 328.

FIGS. 27C and 27D illustrate a distal portion of the catheters of the thermal system 570. As shown, the heated element 304 can be disposed in the lumen 305 of the distal balloon catheter 328. In some variants, the heated element 304 can be potted in the lumen 305 of the distal balloon catheter 328. The heated element 304 can plug the distal end of the lumen 305, which can impede gas and/or fluid flowing through the distal balloon catheter 328 to the distal balloon 320 from escaping through a distal opening of the lumen 305. The heated element 304 can be heated at least to the temperatures described herein. The temperature of the heated element 304 can be altered, which can include being altered based on sensed conditions at the occlusion site (e.g., temperature at the occlusion site). The heated element 304 can be heated during penetration and/or crossing over of an occlusion. The heated element 304 may, in some variants, not be heated once penetration and/or crossing over of the occlusion have been accomplished. In some variants, the heated element 304 can be fixed in position relative to the distal balloon catheter 328. In some variants, the heated element 304 can be retracted into the lumen 305 of the distal balloon catheter 328 and/or deployed distally out from the lumen 305 of the distal balloon catheter 328. The heated element 304 can include a wire that extends distally, loops, and returns proximally. The heated element 304, in some variants, can be removed proximally from the lumen 305 of the distal balloon catheter 328.

FIG. 27E illustrates a cross-sectional view of the multi-lumen tube 572 with the irrigation catheter 308 and distal balloon catheter 328. The multi-lumen tube 572 can have an outer diameter of various sizes, which can at least include less than 14 Fr, 14 Fr, or greater than 14 Fr. As shown, the multi-lumen tube 572 can include a proximal balloon lumen 612. The proximal balloon lumen 612 can be various sizes, which can include less than 1.3 millimeters (e.g., 4 Fr), 1.3 millimeters (e.g., 4 Fr), or larger than 1.3 millimeters (e.g., 4 Fr). As described herein, a distal end of the proximal balloon lumen 612 can be plugged to prevent escape of the fluid and/or gas flowing through the proximal balloon lumen 612 to inflate and/or deflate the proximal balloon 322. In some variants, a guidewire can be disposed through the proximal balloon lumen 612, which may seal the distal end of the proximal balloon lumen 612 to impede the escape of gas and/or fluid for inflating and/or deflating the proximal balloon 322.

The aspiration lumen 610 of the multi-lumen tube 572 can be used to aspirate matter (e.g., heated fluid, softened and/or emulsified thrombus and/or plaque, and/or other material) from an occlusion site. The aspiration lumen 610 can be a working lumen. In some variants, the aspiration lumen 610 can be braided. The irrigation catheter 308 and/or distal balloon catheter 328 can be disposed inside the aspiration lumen 610. The distal balloon catheter 328 can be disposed inside of a lumen 311 of the irrigation catheter 308. Heated fluid can flow in the lumen 311 of the irrigation catheter 308 between the distal balloon catheter 328 and an inner wall of the irrigation catheter 308 defining the lumen 311. The distal end of the lumen 311 of the irrigation catheter 308 can be open to enable the distal balloon catheter 328 to be advanced distally out of the irrigation catheter 308. Heated fluid can flow through the lumen 311 and out of the one or more apertures 324 and/or open distal end to an occlusion site to soften and/or emulsify an occlusion. Fluid and/or gas to inflate and/or deflate the distal balloon 320 can flow through a lumen 305 of the distal balloon catheter 328. The distal end of the lumen 305 can be plugged (e.g., closed) to prevent escape of the fluid and/or gas. The distal end of the lumen 305, in some variants, can be plugged by the heated element 304. The heated element 304 can be disposed in the lumen 305 of the distal balloon catheter 328.

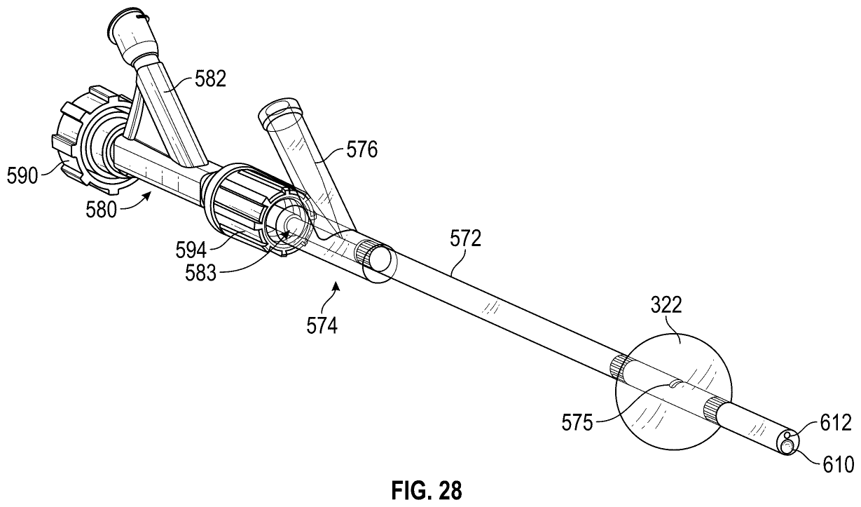

FIG. 28 illustrates an aspiration and working channel port 583. The aspiration and working channel port 583 can be coupled to the Y connector 574 to fluidically couple with the aspiration lumen 610 of the multi-lumen tube 572. The irrigation catheter 308 and/or distal balloon catheter 328 can be disposed through the aspiration and working channel port 583 to access the aspiration lumen 610.

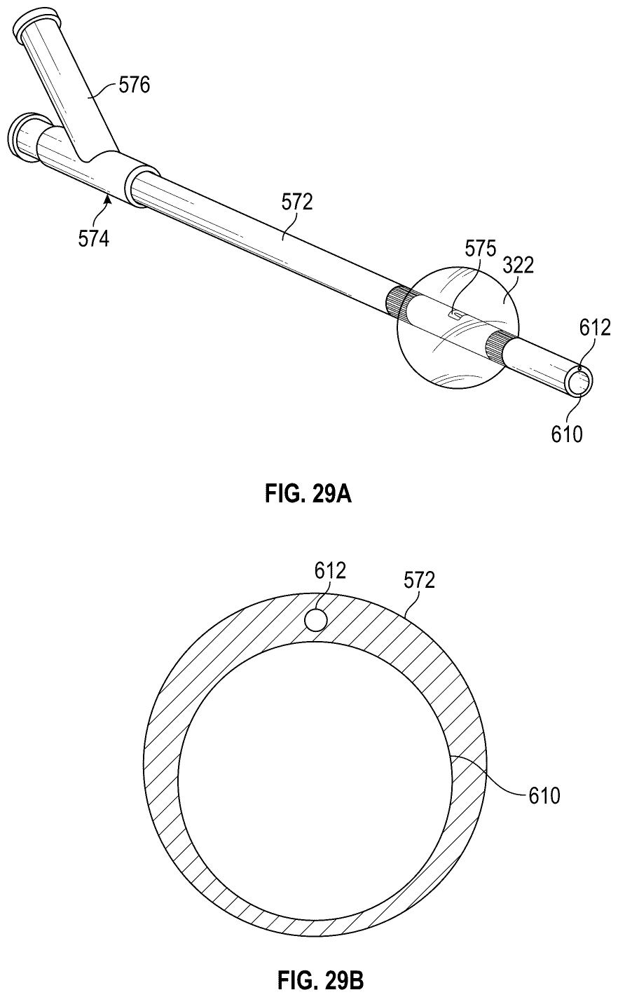

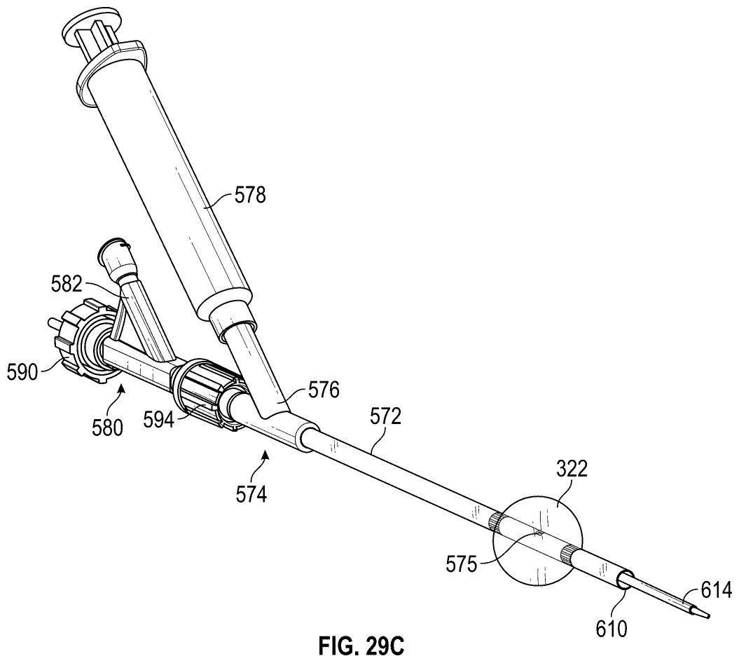

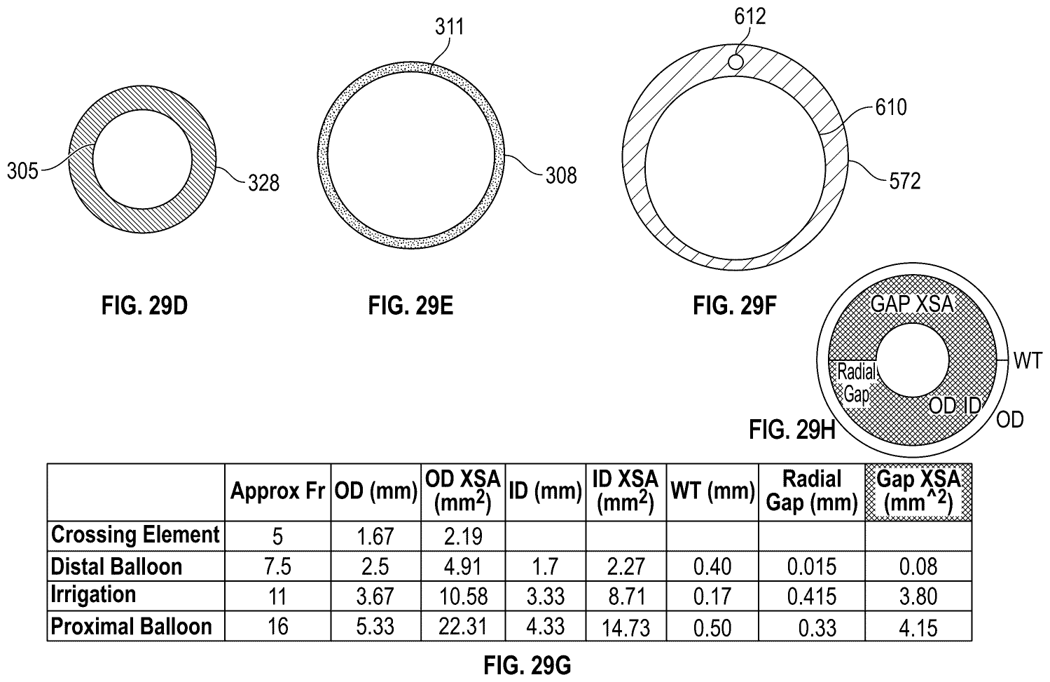

FIG. 29A illustrates the multi-lumen tube 572 and Y connector 574 separate from the remainder of the catheters and connectors of the thermal system 570. FIG. 29B illustrates a cross-sectional view of the multi-lumen tube 572 to show the proximal balloon lumen 612 and aspiration lumen 610. The size of the aspiration lumen 610 relative to the size of the proximal balloon lumen 612 shown in FIG. 29A can be greater compared to the example shown in FIG. 27E. FIG. 29C illustrates a guidewire 614 disposed through the aspiration lumen 610 of the multi-lumen tube 572. As described herein, the guidewire 614 can be navigated to an occlusion site. The multi-lumen tube 572 can be advanced over the guidewire 614 to proximal of the occlusion site. In some variants, the irrigation catheter 308 and/or distal balloon catheter 328 can be advanced over the guidewire 614 to the occlusion site. In some variants, the guidewire 614 can be retracted prior to navigating the irrigation catheter 308 and/or distal balloon catheter 328 through the aspiration lumen 610 to the occlusion site. The guidewire 614 can be various sizes, which can include 5 Fr.

FIG. 29D illustrates a cross-section view of the distal balloon catheter 328 showing the lumen 305 of the distal balloon catheter 328. FIG. 29E illustrates a cross-section view of the irrigation catheter 308 showing the lumen 311 of the irrigation catheter 308. FIG. 29F illustrates a cross-section view of the multi-lumen tube 572 showing the proximal balloon lumen 612 and aspiration lumen 610.

FIG. 29G illustrates example dimensions for the heated element 304 (e.g., crossing element), distal balloon 320, proximal balloon 322, and irrigation catheter 308. The example dimensions throughout this disclosure are exemplary and should not be considered limiting.

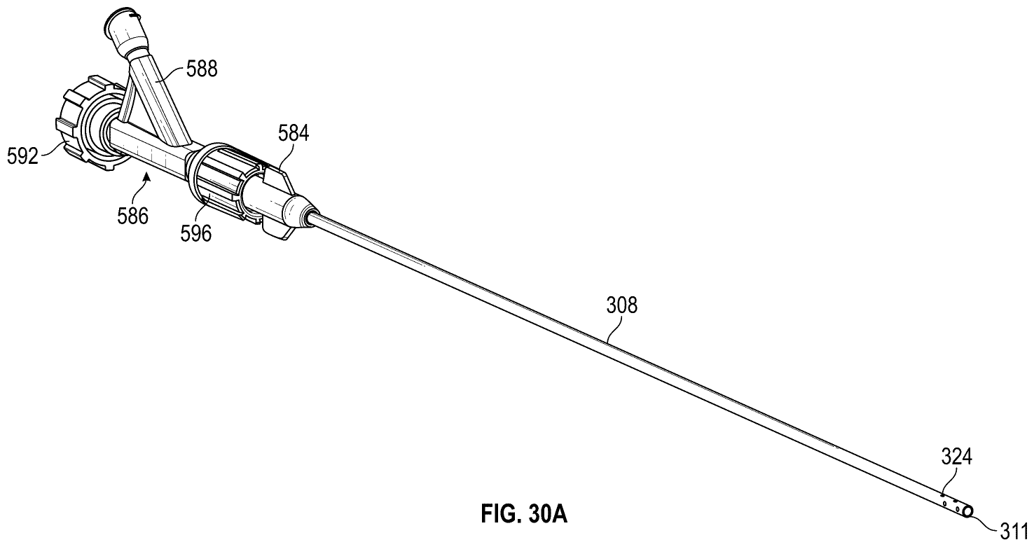

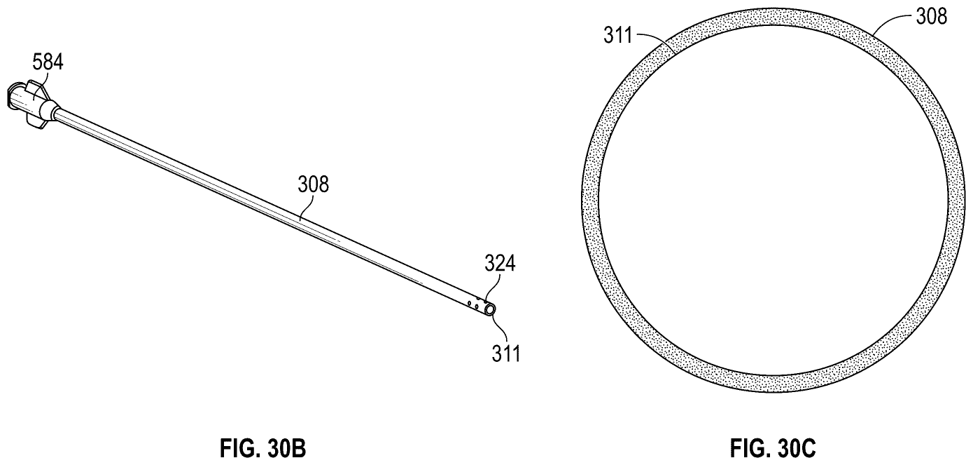

FIG. 30A illustrates the irrigation catheter 308 outside of the multi-lumen tube 572 with the irrigation catheter hub 584 coupled to the Y connector 586 by way of the connector 596. The irrigation catheter 308 can be varying lengths to reach different locations. The one or more apertures 324 can be disposed at a distal portion (e.g., distal end) of the irrigation catheter 308. The one or more apertures 324 can be formed with a variety of techniques, which can at least include laser-cutting or punching. FIG. 30B illustrates the irrigation catheter 308 with the irrigation catheter hub 584 decoupled from the Y connector 586. FIG. 30C illustrates a cross-section view of the irrigation catheter 308.

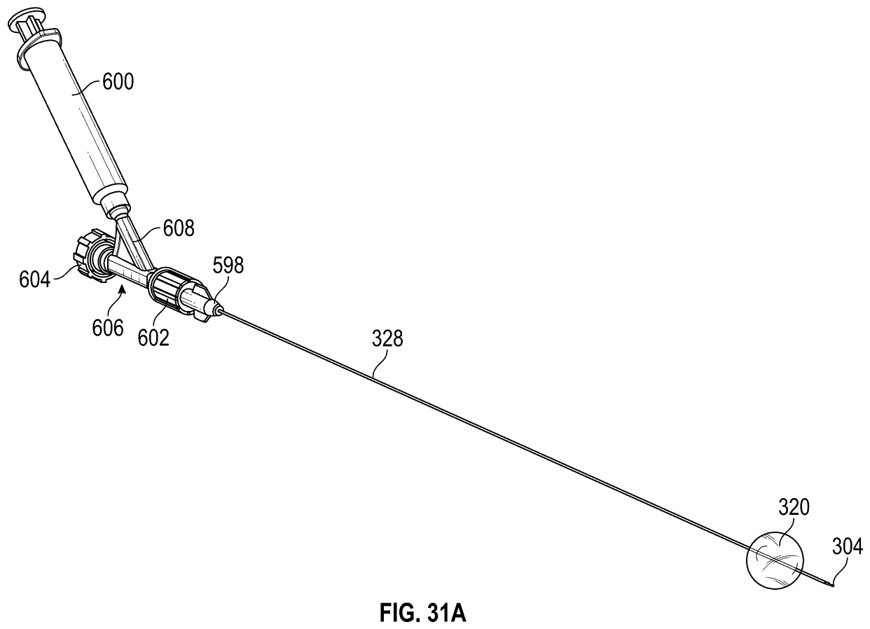

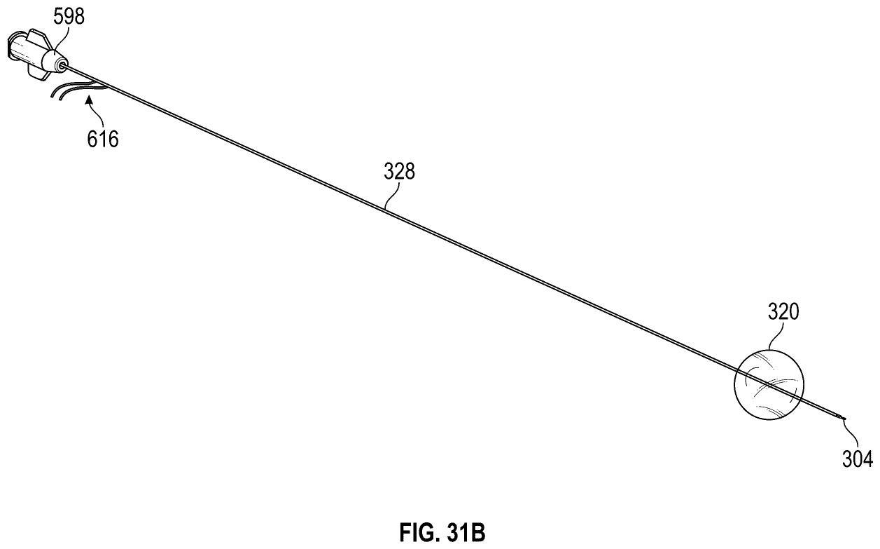

FIG. 31A illustrates the distal balloon catheter 328 outside of the multi-lumen tube 572 with the distal balloon catheter hub 598 coupled to the Y connector 606 by way of the connector 602. The distal balloon catheter 328 can be varying lengths to reach different locations. FIG. 31B illustrates the distal balloon catheter 328 with the distal balloon catheter hub 598 decoupled from the Y connector 606. As shown, the heated element 304 can include wiring 616 extending through the distal balloon catheter 328 to the heated element 304. For example, the wires 616 can extend through the lumen 305 of the distal balloon catheter 328. The wires 616 can direct current to the heated element 304 to adjust the temperature of the heated element 304 (e.g., heat). The wires 616 can exit the distal balloon catheter 328 distal of the distal balloon catheter hub 598.

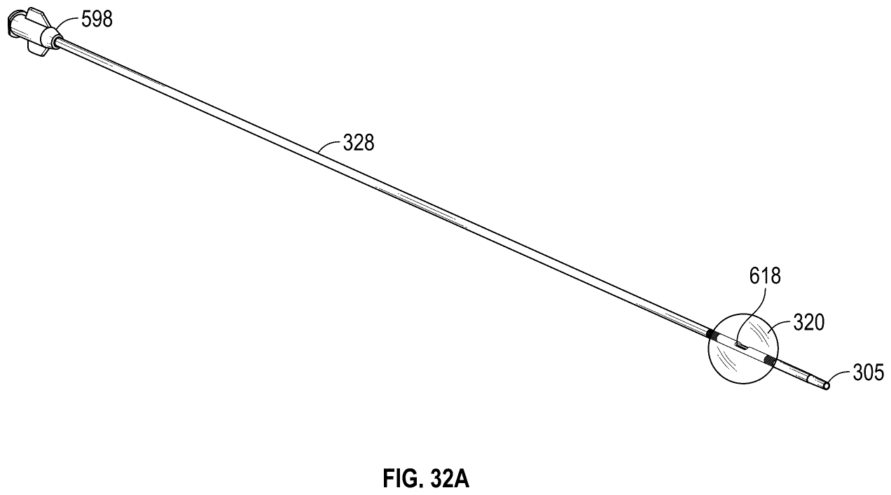

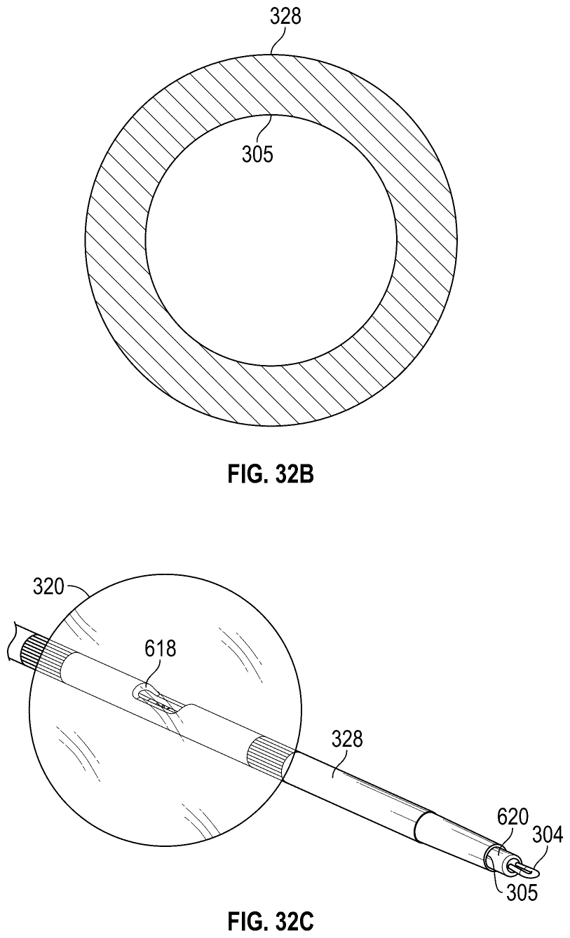

FIG. 32A illustrates the distal balloon catheter 328 outside of the multi-lumen tube 572. As shown, the distal balloon catheter 328 can include an aperture 618 (e.g., opening, cutout, hole) through which gas and/or fluid can flow to inflate or deflate the distal balloon 320. The aperture 618 can open into the distal balloon 320. The aperture 618 can provide access into the lumen 305, as shown in the cross section of the distal balloon catheter 328 in FIG. 32B, through which the gas and/or fluid can flow. FIG. 32C illustrates a member 620 (e.g., cylinder, rod, shaft) with the heated element 304 disposed on a distal end thereof. The member 620 can be disposed in the lumen 305 of the distal balloon catheter 328. The member 620 can plug the distal end of the lumen 305 of the distal balloon catheter 328, which can impede escape of the gas and/or fluid used to inflate or deflate the distal balloon 320. The member 620 can be translated (e.g., slid) distally and/or proximally to deploy and/or stow the member 620 and heated element 304. For example, the member 620 can be translated distally to position the heated element 304 outside of the lumen 305 of the distal balloon catheter 328. The heated element 304 can be heated by at least the techniques described herein. For example, current can be applied to the heated element 304 by way of the wires 616. The distal balloon catheter 328 with the heated element 304 exposed can be advanced to penetrate and/or crossover an occlusion. The heated element 304 (e.g., member 620 and heated element 304) can be retracted into the lumen 305 of the distal balloon catheter 328. In some variants, the member 620 can be at a fixed position relative to the distal balloon catheter 328, which can include a fixed position with the member 620 protruding from the lumen 305 of the distal balloon catheter 328 to expose the heated element 304. The member 620 can be various sizes, which can at least include less than 5 Fr (e.g., 1.5 millimeters), about 5 Fr (e.g., 1.5 millimeters), or more than 5 Fr (e.g., 1.5 millimeters).

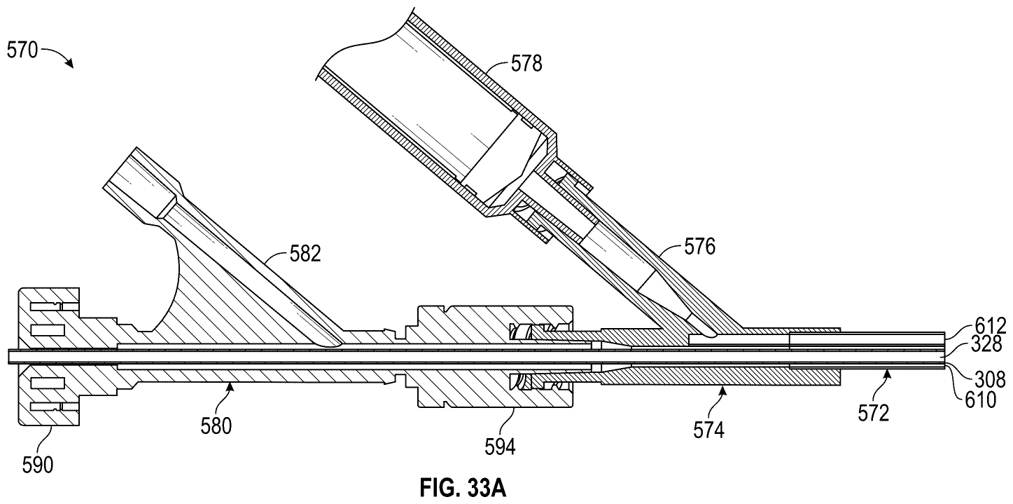

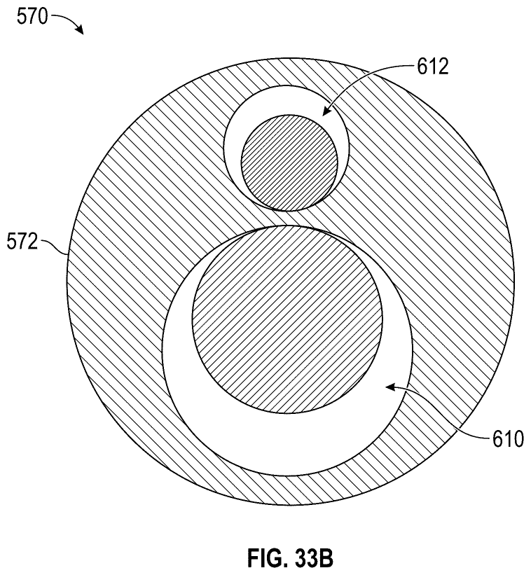

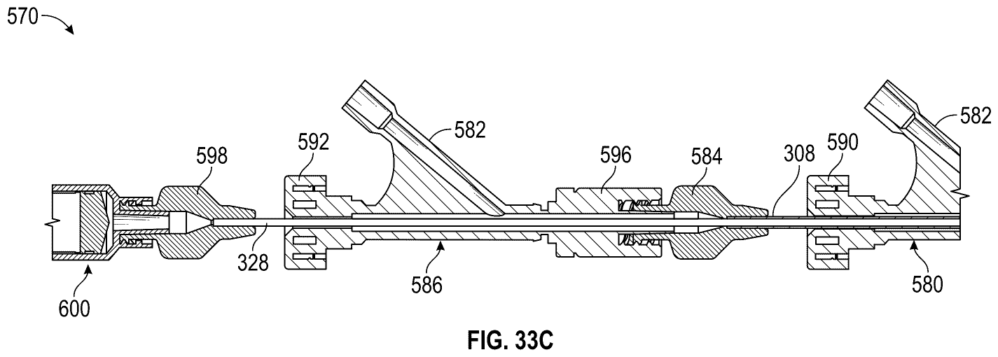

FIG. 33A illustrates a cross-section view of the multi-lumen tube 572, Y connector 574, connector 594, Y connector 580, linear actuator 590, irrigation catheter 308, and distal balloon catheter 328. FIG. 33B illustrates a cross-section view of the multi-lumen tube 572 to show the proximal balloon lumen 612 and aspiration lumen 610. FIG. 33C illustrates a cross-section view of the Y connector 580, linear actuator 590, irrigation catheter hub 584, connector 596, Y connector 586, linear actuator 592, distal balloon catheter hub 598, syringe 600, irrigation catheter 308, and distal balloon catheter 328.

In use, a guidewire 614 can be navigated to proximal of an occlusion (e.g., thrombus and/or plaque), which can be partial or complete. The multi-lumen tube 572 can be advanced over the guidewire 614 to proximal of the occlusion with the guidewire 614 disposed through the aspiration lumen 610. The irrigation catheter 308 can be advanced in the aspiration lumen 610 over the guidewire 614 to proximal of the occlusion. The guidewire 614 can be removed. The distal balloon catheter 328 can be advanced through the lumen 311 of the irrigation catheter 308 positioned in the aspiration lumen 610 of the multi-lumen tube 572. In some variants, the distal balloon catheter 328 can be advanced through the aspiration lumen 610 but outside of the irrigation catheter 308. In some variants, the distal balloon catheter 328 can be advanced through another lumen of the multi-lumen tube 572. The distal balloon catheter 328 can be advanced to a position distal of the distal end of the multi-lumen tube 572. The proximal balloon 322 can be inflated. For example, the syringe 578 can urge gas and/or fluid through the proximal balloon lumen 612 of the multi-lumen tube 572 and out the aperture 575 into the proximal balloon 322. The proximal balloon 322 can expand to contact the surrounding wall of the blood vessel to anchor the multi-lumen tube 572 in position. The heated element 304 can be heated, which can include applying a current to the heated element 304. In some variants, the member 620 can be advanced to position the heated element 304 outside of the lumen 305 of the distal balloon catheter 328. The heated element 304 can be advanced to penetrate and/or crossover the occlusion. The heat from the heated element 304 can ease penetration and/or crossing over, which can include softening and/or emulsifying the portions of the occlusion contacted by the heated element 304. The distal balloon catheter 328 can be advanced with the heated element 304 such that the distal balloon catheter 328 penetrates and/or crosses over the occlusion. The temperature of the heated element 304 can be lowered after the occlusion has been penetrated and/or crossed. For example, the heated element 304 can cease being heated (e.g., no electrical current applied) after the occlusion has been penetrated and/or crossed.

The distal balloon catheter 328 can be advanced to position the distal balloon 320 distal of the occlusion. The distal balloon catheter 328 can be inflated. For example, the syringe 600 can urge gas and/or fluid through the lumen 305 of the distal balloon catheter 328 and out the aperture 618 into the distal balloon 320. The distal balloon 320 can expand to contact the surrounding wall of the blood vessel. The expanded distal balloon 320 and proximal balloon 322 can isolate (e.g., fluidically isolate) the segment of the blood vessel between the distal balloon 320 and proximal balloon 322.

The irrigation catheter 308 can be advanced to position the one or more apertures 324 at the occlusion. Heated fluid can be delivered to the occlusion by way of the one or more apertures 324 and/or an open distal end of the irrigation catheter 308. For example, a heated fluid, which can be from a reservoir, can be introduced through the irrigation port 588 of the Y connector 586 into the irrigation catheter 308. The heated fluid can soften and/or emulsify the occlusion. The fluid can be delivered to the occlusion at least at the temperatures described herein. The temperature, flow rate, pressure, and/or other characteristics of the fluid can be altered, which can include being altered based on conditions (e.g., temperature, pressure, etc.) in the blood vessel, at the occlusion, and/or within any of the components of the thermal system 570. The heated fluid can be pumped through the irrigation catheter 308 with one or more pumps.

The aspiration lumen 610 of the multi-lumen tube 572 can aspirate the softened and/or emulsified occlusion, heated fluid, and/or other material. The rate of aspiration through the aspiration lumen 610 can be altered, which can include being altered based on conditions (e.g., temperature, pressure, flow rate, etc.) in the blood vessel, at the occlusion, within the aspiration lumen 610, and/or any component of the thermal system 570. The thermal system 570 can include one or more sensors (e.g., temperature sensor, pressure sensor, flow rate sensor, etc.) to sense conditions in the blood vessel, at the occlusion, and/or within any component of the thermal system 570. The aspirated matter can flow through the aspiration lumen 610 and out the aspiration port 582 of the Y connector 580. The aspirated matter can flow into a tank or the like. The aspirated matter can be pumped through the aspiration lumen 610 with one or more pumps for removal. In some variants, the thermal system 570 can include a separate aspiration catheter that can be advanced through a lumen of the multi-lumen tube 572.

With irrigation and/or aspiration stopped (e.g., when the occlusion is removed), the distal balloon 320 and proximal balloon 322 can be deflated. For example, the gas and/or fluid in the distal balloon 320 and/or proximal balloon 322 can be removed. For example, the syringe 578 can urge gas and/or fluid in the proximal balloon 322 through the aperture 575 into the proximal balloon lumen 612 and out the port 576 of the Y connector 574 back into the syringe 578. The syringe 600 can urge gas and/or fluid in the distal balloon 320 through the aperture 618 into the lumen 305 of the distal balloon catheter 328 and back into the syringe 600, which can include by way of the port 608 of the Y connector 606. The distal balloon catheter 328, irrigation catheter 308, and/or multi-lumen tube 572 can be retracted for removal. In some variants, the distal balloon catheter 328 and irrigation catheter 308 can be retracted back into the multi-lumen tube 572 and the distal balloon catheter 328, irrigation catheter 308, and multi-lumen tube 572 can be retracted together with the distal balloon catheter 328 and irrigation catheter 308 disposed in the multi-lumen tube 572.

In some variants, the distal balloon catheter 328 can be positioned with the distal balloon 320 distal of the occlusion and/or the irrigation catheter 308 positioned proximal and/or at (e.g., in) the occlusion prior to positioning a distal end of the multi-lumen tube 572 proximal of the occlusion. In some variants, the heated element 304 (e.g., member 620 with the heated element 304) can be inserted through a proximal opening of the distal balloon catheter 328, which can include the distal balloon catheter hub 598, and advanced distally out of the lumen 305 of the distal balloon catheter 328.

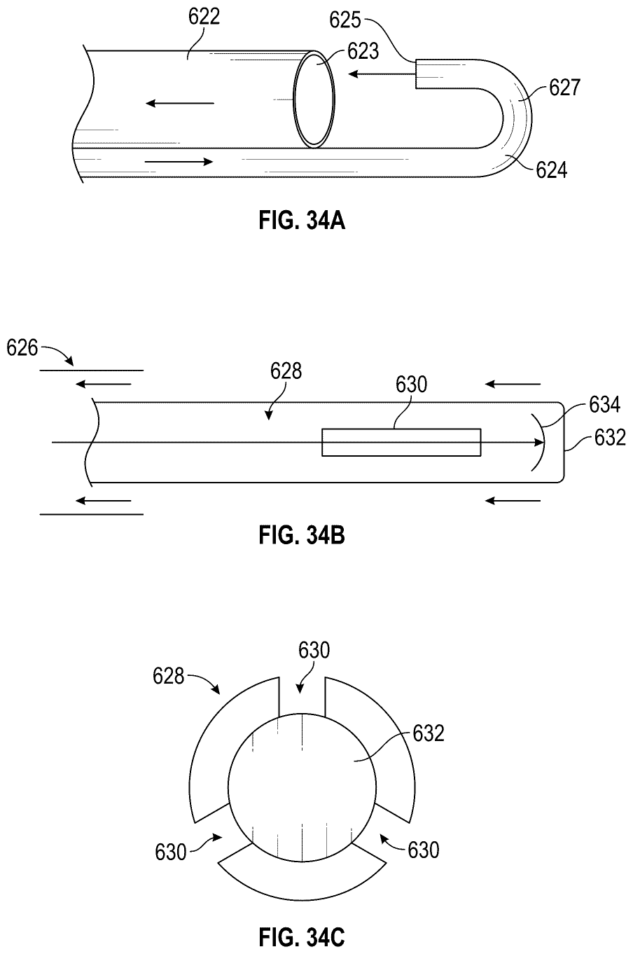

In some variants, irrigation of heated fluid (e.g., a jet of heated fluid) and aspiration can be used to penetrate and/or crossover an occlusion (e.g., thrombus and/or plaque), which can replace or be used in conjunction with the heated element 304. In some variants, irrigation of heated fluid (e.g., a jet of heated fluid) and aspiration can be used to soften and/or emulsify an occlusion for removal by way of aspiration. In some variants, irrigation of heated fluid (e.g., a jet of heated fluid) can be used to soften and/or emulsify an occlusion, which may not include aspiration. FIG. 34A illustrates an example irrigation catheter 624 and aspiration catheter 622, which can be used to penetrate and/or crossover an occlusion and/or soften and/or emulsify an occlusion for aspiration. As shown, the irrigation catheter 624 can include a curve 127 (e.g., bend, hook, turn) such that an opening 625 (e.g., open end, open distal end) faces in a proximal direction. The irrigation catheter 624 can extend distally and curve proximally at the curve 127 to face the opening 625 in the proximal direction. The aspiration catheter 622 can include an opening (e.g., open end, open distal end) that faces in the distal direction. The opening 625 of the irrigation catheter 624 can face toward the opening 623 of the aspiration catheter 622. The central axis of the opening 625 of the irrigation catheter 624 can extend through the opening 623 of the aspiration catheter 622. In use, the irrigation catheter 624 and the aspiration catheter 622 can be navigated to proximal of an occlusion using at least the techniques described herein. Heated fluid can flow through the irrigation catheter 624 and out the opening 625. The irrigation catheter 624 can be advanced to penetrate and/or crossover the occlusion. The heated fluid can ease penetration and/or crossover, which can include softening and/or emulsifying the occlusion. The aspiration catheter 622 can be advanced with the irrigation catheter 624 to maintain a gap within a range between the opening 625 and the opening 623. In some variants, the irrigation catheter 624 and aspiration catheter 622 can be fixed together to maintain a relative positioning. The aspiration catheter 622 can aspirate heated fluid, softened and/or emulsified occlusion, and/or other material. With the opening 625 facing the opening 623, the heat from the heated fluid can be localized. In some variants, the irrigation catheter 624 and/or aspiration catheter 622 can be used to remove the occlusion, which can be in addition to penetrating and/or crossing.

FIGS. 34B and 34C illustrate an irrigation device 628 (e.g., irrigation catheter) that can be used to penetrate and/or crossover an occlusion (e.g., thrombus and/or plaque) and/or soften and/or emulsify the occlusion for removal. The irrigation device 628 can include a closed distal end 632, which can include a flat outer surface (e.g., include a flat surface that is perpendicular to the axis of the irrigation device 628) and/or a curved inner surface 634. The irrigation device 628 can include one or more openings 630 (e.g., apertures), such as one, two, three, four, five, six or more. The one or more openings 630 can be disposed in the peripheral wall of the irrigation device 628 that defines an internal lumen. The one or more openings 630 can be disposed circumferentially about an axis of the irrigation device 628 (e.g., axis of the internal lumen). The one or more openings 630 can be disposed proximate the distal end 632. The heated fluid can flow distally through the internal lumen and out the one or more openings 630 to the occlusion. The heated fluid can flow distally through the internal lumen to the closed distal end 632 and be deflected by the curved surface 634 and out the one or more openings 630 to the occlusion. The one or more openings 630 can be various sizes and/or shapes. The one or more openings 630 can be elongate in the longitudinal direction of the irrigation device 628. The irrigation device 628 can be deployed from inside of an aspiration device 626 (e.g., aspiration catheter). For example, the irrigation device 628 can be advanced distally through an opening of the aspiration device 626. The irrigation device 628 can soften and/or emulsify the occlusion with heated fluid flowing through the one or more openings apertures 630 to facilitate penetration and/or crossover and/or softening and/or emulsification for removal by way of aspiration. The aspiration device 626 can aspirate the heated fluid, softened and/or emulsified lens, and/or other matter through the opening of the aspiration device 626 for removal. The aspiration device 626 can have a lower pressure inside that sucks the heated fluid, softened and/or emulsified lens, and/or other matter through the opening of the aspiration device 626 for removal. The low pressure inside the aspiration device 626 can localize the impact of the heated fluid. The irrigation device 628 and/or aspiration device 626 can be used to remove the occlusion, which can be in addition to penetrating and/or crossing.

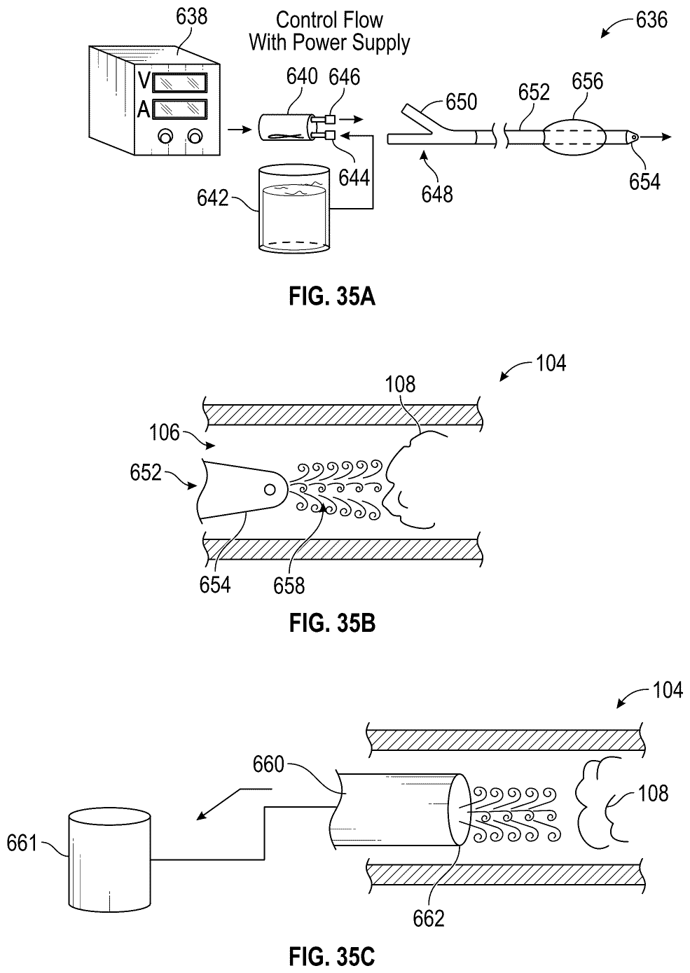

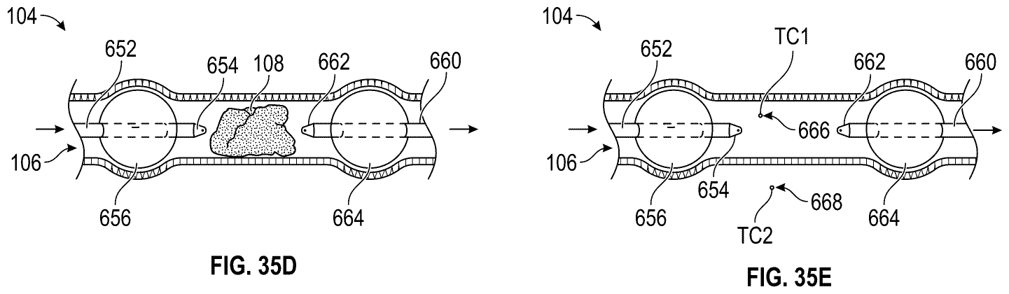

FIG. 35A illustrates a fluid jet system 636 (e.g., thermal penetration system, thermal crossover system, thermal system, thermal fluid system) that can be used to penetrate, crossover, and/or remove an occlusion (e.g., thrombus and/or plaque). The fluid jet system 636, in some variants, can break up (e.g., fragment) an occlusion with a jet (e.g., stream) of fluid (e.g., heated fluid). The use of heated fluid can soften and/or emulsify the occlusion, which can ease fragmentation. The fluid jet system 636 can include a fluid jet catheter 652 with a distal end 654 (e.g., open end, open distal end, opening, nozzle, outlet, etc.). The distal end 654 can be navigated to proximal of an occlusion. The fluid jet catheter 652 can have a balloon 656 that can be expanded and deflated. When expanded, the balloon 656 can contact the surrounding wall of the blood vessel to impede flow. The fluid jet catheter 652 can be coupled with a pump 640 (e.g., DC pump). The pump 640 can be coupled with a control device 638 to control the pump 640, which can include powering the pump. The pump 640 can pump fluid from a tank 642 (e.g., reservoir, bag, bottle, container, syringe, chamber, vessel, compartment, etc.) through an inlet 644 and into the fluid jet catheter 652 by way of an outlet 646. The fluid, in some variants, can be heated in the tank 642. The fluid jet catheter 652 can be coupled to the outlet 646 of the pump 640 with a Y connector 648. The Y connector 648 can include a port 650. In some variants, matter (e.g., heated fluid, softened and/or emulsified occlusion, and/or other material) can be aspirated through the fluid jet catheter 652 and out the port 650 of the Y connector 648. For example, a pump and/or vacuum source can be fluidically coupled to the port 650 to urge matter (e.g., heated fluid, softened and/or emulsified occlusion, and/or other material) out by way of the port 650. The fluid jet catheter 652 can include one or more openings to facilitate aspiration. In some variants, the distal end 654 can include an opening for aspiration. In some variants, heated fluid can flow out of the distal end 654 and then matter can be aspirated through the distal end 654. In some variants, an aspiration device can be introduced to aspirate matter. In some variants, matter is not aspirated. In some variants, the balloon 656 is not included.

FIG. 35B illustrates a fluid jet 658 (e.g., stream) flowing at thrombus 108 for fragmentation, which can include softening and/or emulsification. The fluid jet 658 can be heated. The temperature, flow rate, size of the opening at the distal end 654, distance to the thrombus 108, flux, pressure, duration the fluid jet system 636 directs the fluid jet 658 at the occlusion (e.g., thrombus 108), and/or other characteristics can be adjusted. The foregoing characteristics can be adjusted based on sensed conditions in the blood vessel (e.g., vein 104), conditions at the occlusion (e.g., thrombus 108), characteristics of the occlusion (e.g., thrombus 108), and/or conditions within the fluid jet system 636. For example, the foregoing characteristics can be adjusted at least based on a temperature at the distal end 654, mass of the thrombus 108, density of the thrombus 108, and/or length of the thrombus 108. In some variants, the heated fluid, fragmented occlusion, softened occlusion, and/or emulsified occlusion may not be aspirated.

FIG. 35C illustrates an aspiration device 660 (e.g., aspiration catheter) aspirating the fragmented thrombus 108 (e.g., softened and/or emulsified thrombus 108) and/or heated fluid through an opening 662 (e.g., open distal end). The aspirated matter can flow into a tank 661 (e.g., reservoir, bag, bottle, container, syringe, chamber, vessel, compartment, etc.). In some variants, the aspiration catheter 660 can aspirate matter while the fluid jet catheter 652 directs the fluid jet 658 at the thrombus 108. In some variants, the aspiration catheter 660 can aspirate matter after the fluid jet catheter 652 directs the fluid jet 658 at the thrombus 108. In some variants, the fluid jet catheter 652 can be deployed through the aspiration catheter 660. In some variants, the aspiration catheter 660 can be deployed through the fluid jet catheter 652. In some variants, the aspiration catheter 660 can be disposed on an opposite side of the thrombus 108 as the fluid jet catheter 652. The vacuum force (e.g., sucking force) of the aspiration catheter 660 can be adjusted, which can include being adjusted based on the conditions and/or characteristics described herein. The size of the opening 662 can be adjusted, which can include being adjusted based on the conditions and/or characteristics described herein.

FIG. 35D illustrates the fluid jet catheter 652 disposed on one side (e.g., proximal side) of the thrombus 108 to emit a fluid jet to break up (e.g., fragment), which can include soften and/or emulsify, the thrombus 108. In some variants, a second fluid jet catheter 660 can be disposed on an opposite side of the thrombus 108 (e.g., distal side of the thrombus 108). The second fluid jet catheter 660 can be oriented to face in a proximal direction. The second fluid jet catheter 660 can include a balloon 664 that can be inflated and/or deflated. With the balloon 656 and balloon 664 inflated, the segment of the blood vessel (e.g., vein 104) between the balloon 656 and balloon 664 can be isolated (e.g., fluidically isolated) from the remainder of the blood vessel. In some variants, the fluid jet catheter 652 can direct a fluid jet, which can be heated, out of the distal end 654 to the thrombus 108. The fluid jet can break up, soften, and/or emulsify the thrombus 108. In some variants, the second fluid jet catheter 660 can direct a fluid jet, which can be heated, out of the distal end 654 to the thrombus 108. In some variants, no aspiration is performed. In some variants, one of or both the catheter 652 and the catheter 660 can direct a fluid jet at the thrombus 108 for breakup (e.g., fragmentation), softening, and/or emulsification. In some variants, one or both the catheter 652 and the catheter 660 can aspirate heated fluid, fragmented thrombus, softened thrombus, and/or emulsified thrombus. As illustrated in FIG. 35E, the fluid jet system 636 can include temperature sensors to sense temperatures in the blood vessel (e.g., vein 104), outside the blood vessel, and/or in the fluid jet system 636. For example, the fluid jet system 636 can include a temperature sensor 666 in the blood vessel, which can include being at the occlusion. The fluid jet system 636 can include a temperature sensor 668 outside the blood vessel. The fluid jet system 636 can adjust the fluid jet and/or aspiration based on the sensed temperatures and/or other sensed characteristics. In some variants, one fluid jet can be used. In some variants, multiple fluid jets can be used. In some variants, peripheral fluid jets can be used. In some variants, aspiration is used. In some variants, aspiration is not used. In some variants, the catheter 652 and/or catheter 660 can include a port (e.g., side port) for aspiration. In some variants, a dual cannula system can be used to facilitate irrigation and aspiration.

The catheters described herein can be cannulas, tubes, etc. In some variants, expandable devices other than balloons can be used in the system and methods described herein, which can at least include bags, umbrellas, meshes, cages, nets, etc. In some variants, the catheters described herein can be referred to as tubes and/or extrusions. In some variants, the systems and/or methods described herein can omit one or both of the proximal and distal balloons. In some variants, micromechanoaspiration and/or microthermomechanoaspiration can be used. In some variants, a reverse jet can be employed to localize emulsification. For example, concentric irrigation and aspiration catheters can be used with the aspiration catheter having a low pressure to suck in the heated fluid, which can localize the softening and/or emulsification zone. In some variants, the aspiration catheter can be disposed proximate an opening of the irrigation catheter such that the low pressure in the aspiration catheter can suck in the heated fluid, which can localize the softening and/or emulsification zone.

In some variants, cutting tools (e.g., mechanical cutting tools, scraping devices, slicing devices), which may at least include a brush, wire brush, wire, coil, lasso, corkscrew, drill, wire, spatula, auger, tapered wire drill, blade, knife, constricting coil, vitrectomy probe, ultrasonic cutter, extendable scrubbers (e.g., wires), umbrella, inverted mesh, and/or others, can be used with any of the systems and/or methods described herein. The cutting tools, in some variants, can include heated elements to soften and/or emulsify an occlusion. In some variants, the irrigation devices and/or aspiration devices (e.g., catheters) described herein can include features (e.g., flanges, fins, coils, protrusions, cutting edges, projections, blades, etc.) to mechanically break up (e.g., fragment) an occlusion. In some variants, the features can include heated elements. For example, the irrigation catheters described herein can include a protrusion disposed on a distal end thereof. The irrigation catheter can be rotated and/or translated such that the protrusion cuts into an occlusion to fragment the occlusion. The protrusion can include a heated element to soften and/or emulsify the occlusion. A wire (e.g., single or multiple) can be deployed to help with mechanical breakup of an occlusion (e.g., chronic occlusion). For example, the wire can be deployed to engage with an occlusion disposed between two inflated balloons. The wire can be moved to break up the occlusion, which can be performed simultaneously with irrigation with the heated fluid. The cutting tools can be made of a variety of materials such as polymers, metals, metal alloys, ceramics, and/or others. The cutting tools can be rotated, advanced distally, and/or retracted proximally to break up (e.g., fragment) an occlusion. The cutting tools can be disposed on the irrigation device, aspiration device, crossing device, a separate device, and/or other devices, which can include being disposed at an end (e.g., distal portion, middle portion, and/or proximal portion). The cutting tools, such as a brush (e.g., wire brush) can be disposed throughout a device. The cutting tools can be a partial brush, flared brush, and/or variable diameter brush.

In some variants, the aspiration devices (e.g., catheters) described herein can include a dual port luer hub. In some variants, the aspiration devices (e.g., catheters) described herein can include a single port luer hub. In some variants, the irrigation devices (e.g., catheters) described herein can include a single port luer hub. In some variants, the crossing devices (e.g., distal balloon catheters, crossing catheters) described herein can include a single port luer hub.

In some variants, the irrigation devices (e.g., irrigation catheters) can include one or more heated elements (e.g., ring(s), spot(s), wire(s), etc.) that can interface with the occlusion to soften and/or emulsify the occlusion. In some variants, the aspiration devices (e.g., aspiration catheters) can include a heated mouth (e.g., heated opening), which can include one or more ring(s), spot(s), wire(s), etc. The heated mouth can soften and/or emulsify the occlusion, which can include easing separation of the occlusion from the wall of the blood vessel, for aspiration. The heated mouth of the aspiration device can be advanced to interface the occlusion. The heated mouth can be used simultaneously and/or separately (e.g., after) irrigation with the heated fluid (e.g., fluid jet). In some variants, the aspiration devices described herein can be advanced with the irrigation devices. In some variants, the aspiration devices described herein can be advanced while aspirating.

In some variants, the fluid jets described herein can be used in conjunction with a heated element for crossing and/or penetration. In some variants, closed-loop techniques and/or artificial intelligence can control (e.g., modulate) the heat (e.g., current), flow rates, pressures, etc. applied by the systems described herein. In some variants, the detection of the type of material, thrombus, plaque, and/or occlusion being removed can be detected (e.g., remotely detected) through data, which can include using closed-loop techniques and/or artificial intelligence. In some variants, the systems and/or methods described herein can monitor a quantity of occlusion collected.

In some variants, the systems and methods described herein can include using a medicament, such as a healing agent (e.g., lytics), anti-inflammatory, and/or endothelial promoting (e.g., hormones) medicament.

In some variants, the systems and methods described herein can be used in a neuro environment.

The system and methods described herein can obtain vascular access using percutaneous techniques. A guidewire can be advanced through the blood vessel system (e.g., venous system or arterial system) to an occlusion site, which can be under fluoroscopic guidance. The system and methods described herein can use fluoroscopic guidance. A catheter system of the thermal systems described herein can be guided over the guidewire. The guidewire can be removed. A fluid delivery system can be coupled to an irrigation catheter. A heated fluid can be infused through the irrigation catheter, which can include at a controlled rate and/or temperature. The temperature and flow rate of the heated fluid can be monitored and adjusted. The heated fluid, softened and/or emulsified occlusion (e.g., thrombus and/or plaque), and/or other matter can be aspirated, which can include being aspirated into a waste reservoir. Angiographic imaging and/or ultrasound can be used to monitor a status of the occlusion. The catheter system can be removed. The vascular access can be closed using standard techniques.

In some variants, a patient may be prescribed medicaments, such as antiplatelet or anticoagulants, to prevent formation of new thrombi and/or plaque.

For non-calcified plaque, the root mean-squared error (RMSE) of the image-based decomposition was estimated to be 0.7%, 1.5%, and 0.3% for water, lipid, and protein contents, respectively. As for the calcified plaques, the RMSE of the 5 mm plaques were estimated to be 5.6%, 5.7%, 0.2%, and 3.1%, for water, lipid, calcium, and protein contents, respectively.

### Terminology

Although the systems and methods have been disclosed in the context of certain embodiments and examples, it will be understood by those skilled in the art that the systems and methods extend beyond the specifically disclosed embodiments to other alternative embodiments and/or uses of the embodiments and certain modifications and equivalents thereof. For example, occlusions are referenced herein. Occlusion can refer to partial and/or complete occlusions. Occlusions can refer to occlusions in an artery and/or vein. Occlusions can refer to a thrombus (e.g., clot), which can include acute and/or chronic. Occlusions can refer to plaque. Various features and aspects of the disclosed embodiments can be combined with or substituted for one another in order to form varying modes. The scope of this disclosure should not be limited by the particular disclosed embodiments described herein.

Methods of using the foregoing system(s) (including device(s), apparatus(es), assembly (ies), structure(s) or the like) are included; the methods of use can include using or assembling any one or more of the features disclosed herein to achieve functions and/or features of the system(s) as discussed in this disclosure. Methods of manufacturing the foregoing system(s) are included; the methods of manufacture can include providing, making, connecting, assembling, and/or installing any one or more of the features of the system(s) disclosed herein to achieve functions and/or features of the system(s) as discussed in this disclosure.

Certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as any subcombination or variation of any subcombination.

Moreover, while operations may be depicted in the drawings or described in the specification in a particular order, such operations need not be performed in the particular order shown or in sequential order, and all operations need not be performed, to achieve the desirable results. Other operations that are not depicted or described can be incorporated in the example methods and processes. For example, one or more additional operations can be performed before, after, simultaneously, or between any of the described operations. Further, the operations may be rearranged or reordered in other implementations. Also, the separation of various system components in the implementations described above should not be understood as requiring such separation in all implementations, and it should be understood that the described components and systems can generally be integrated together in a single product or packaged into multiple products. Additionally, other implementations are within the scope of this disclosure.

Conditional language, such as “can,” “could,” “might,” or “may,” unless specifically stated otherwise, or otherwise understood within the context as used, is generally intended to convey that certain embodiments include or do not include, certain features, elements, and/or steps. Thus, such conditional language is not generally intended to imply that features, elements, and/or steps are in any way required for one or more embodiments.

Conjunctive language, such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Some embodiments have been described in connection with the accompanying drawings. Components can be added, removed, and/or rearranged. Orientation references such as, for example, “top” and “bottom” are for ease of ease of discussion and may be rearranged such that top features are proximate the bottom and bottom features are proximate the top. Further, the disclosure herein of any particular feature, aspect, method, property, characteristic, quality, attribute, element, or the like in connection with various embodiments can be used in all other embodiments set forth herein. Additionally, it will be recognized that any methods described herein may be practiced using any device suitable for performing the recited steps.

In summary, various embodiments and examples of juicing devices and methods have been disclosed. Although the systems and methods have been disclosed in the context of those embodiments and examples, it will be understood by those skilled in the art that this disclosure extends beyond the specifically disclosed embodiments to other alternative embodiments and/or other uses of the embodiments, as well as to certain modifications and equivalents thereof. This disclosure expressly contemplates that various features and aspects of the disclosed embodiments can be combined with, or substituted for, one another. Accordingly, the scope of this disclosure should not be limited by the particular disclosed embodiments described above, but should be determined only by a fair reading of the claims that follow.

What is claimed is:

1. A thermal system for removing an occlusion from a blood vessel, the thermal system comprising:
an irrigation catheter comprising one or more apertures at a distal portion;
wherein the irrigation catheter is configured to be navigated through a blood vessel to deliver a heated fluid to an occlusion to soften and/or emulsify the occlusion.

2. The thermal system of claim 1, further comprising an aspiration catheter configured to aspirate the softened and/or emulsified occlusion.

3. The thermal system of claim 2, wherein the irrigation catheter and the aspiration catheter are configured to be concentrically positioned.

4. The thermal system of claim 2, wherein the irrigation catheter is configured to be disposed inside the aspiration catheter.

5. The thermal system of claim 2, wherein the aspiration catheter is configured to be disposed inside the irrigation catheter.

6. The thermal system of claim 1, wherein the one or more apertures of the irrigation catheter are disposed through a peripheral wall of the irrigation catheter.

7. The thermal system of claim 1, wherein the irrigation catheter comprises one or more heated elements to heat the heated fluid.

8. The thermal system of claim 1, further comprising a crossing element configured to be heated to facilitate penetrating the occlusion.

9. The thermal system of claim 1, further comprising a distal balloon configured to be inflated distal of the occlusion.

10. The thermal system of claim 9, further comprising a proximal balloon configured to be inflated proximal of the occlusion.

11. The thermal system of claim 10, wherein the proximal balloon is disposed on an aspiration catheter.

12. The thermal system of claim 1, further comprising a temperature sensor configured to sense a temperature of the heated fluid, wherein the thermal system is configured to adjust the temperature of the heated fluid based on the sensed temperature.

13. The thermal system of claim 1, wherein the occlusion comprises a thrombus.

14. A thermal system for removing an occlusion from a blood vessel, the thermal system comprising:
a multi-lumen tube comprising a proximal balloon and an aspiration lumen, the proximal balloon configured to be inflated proximally of an occlusion;
an irrigation catheter configured to be advanced through and distally out of the multi-lumen tube to the occlusion; and
a distal-balloon catheter comprising a distal balloon configured to be inflated distally of the occlusion, the distal-balloon catheter configured to be advanced through and distally out of the multi-lumen tube;
wherein the irrigation catheter is configured to deliver heated fluid to the occlusion to soften and/or emulsify the occlusion; and
wherein the aspiration lumen is configured to aspirate the softened and/or emulsified occlusion.

15. The thermal system of claim 14, wherein the distal-balloon catheter is advanced through the irrigation catheter in the aspiration lumen.

16. The thermal system of claim 14, further comprising a heated element configured to penetrate the occlusion for crossover.

17. The thermal system of claim 16, wherein the heated element is configured to be advanced through and distally out of the distal-balloon catheter.

18. The thermal system of claim 14, further comprising a temperature sensor configured to sense a temperature of the heated fluid, where the thermal system is configured to adjust the temperature of the heated fluid based on the sensed temperature.

19. A method of applying heated fluid to an occlusion for removal, the method comprising:
proximally positioning a multi-lumen tube relative to an occlusion;
inflating a proximal balloon of the multi-lumen tube;
advancing a distal-balloon catheter through the multi-lumen tube to position a distal balloon distal of the occlusion;
inflating the distal balloon;
advancing an irrigation catheter through the multi-lumen tube to the occlusion;
delivering heated fluid by way of the irrigation catheter to the occlusion to soften and/or emulsify the occlusion; and
aspirating the softened and/or emulsified occlusion.

20. The method of claim 19, further comprising penetrating the occlusion with a heated element to facilitate crossover.
